(19) United States
(12) Patent Application Publication
(10) Pub. No.: US 2026/0130973 A1
(43) Pub. Date: May 14, 2026

(54) COMPOSITIONS AND METHODS FOR DEGRADATION OF LIPOFUSCIN CYCLORETINAL BY MSP1

(71) Applicant: THE TEXAS A&M UNIVERSITY SYSTEM, College Station, TX (US)

(72) Inventors: Coran WATANABE, College Station, TX (US); Vishruth GOWDA, College Station, TX (US); Irum PERVEEN, College Station, TX (US); Brendan FOLEY, College Station, TX (US); Brett JOHNSON, College Station, TX (US)

(21) Appl. No.: 19/118,980

(22) PCT Filed: Oct. 6, 2023
(86) PCT No.: PCT/US2023/076270

§ 371 Date: Apr. 7, 2025

Related U.S. Application Data
(60) Provisional application No. 63/414,020, filed on Oct. 7, 2022.

(51) Int. Cl. A61K 38/44

(52) U.S. Cl. CPC A61K 38/443; C12Y 111/01007

(57) ABSTRACT

The present disclosure provides pharmaceutical compositions comprising i) a MsP1 enzyme and ii) one or more pharmaceutically acceptable carriers. The disclosure also provides methods comprising the pharmaceutical compositions for use in treating patients with various disease states, as well as related sequences and expression vectors.

### CROSS-REFERENCE TO RELATED APPLICATIONS

This application claims the benefit under 35 USC § 119(e) of U.S. Provisional Application Ser. No. 63/414,020, filed on Oct. 7, 2022, the entire disclosure of which is incorporated herein by reference.

### REFERENCE TO SEQUENCE LISTING SUBMITTED ELECTRONICALLY

The official copy of the sequence listing is submitted electronically via EFS-Web as an ASCII formatted sequence listing with a file named 78090-395653_SL created on Oct. 5, 2023 and having a size of 18.8 kilobytes and is filed concurrently with the specification. The sequence listing comprised in this ASCII formatted document is part of the specification and is herein incorporated by reference in its entirety.

### BACKGROUND AND SUMMARY OF THE INVENTION

Age-related macular degeneration (AMD) is a debilitating eye disease that tends to affect people over the age of 55. Lipofuscins are auto-fluorescent, toxic byproducts of the visual cycle that are believed to contribute toward the progression of the AMD.

Development of drugs to inhibit lipofuscin formation has been stymied as studies evaluating the biosynthesis of key lipofuscins such as cycloretinal (also known as all-trans retinal dimer) or A2E. However, studies have not yet identified a specific target enzyme or even a pathway. Therefore, there exists a need for new approaches for targeting reduction of lipofuscin and for early treatment of AMD.

Accordingly, the present disclosure provides compositions and methods that target the accumulation of lipofuscin through catabolism may serve as a method for early treatment of AMD. In particular, the present disclosure provides compositions directed to an enzymatic approach that is capable of degrading the lipofuscin. The compositions and methods described herein utilize a peroxidase enzyme from the organism Marasmius scorodonius (MsP1). Importantly, the compositions and methods of the present disclosure demonstrate the ability to degrade this toxic metabolite into non-toxic byproducts. As a result, the present disclosure provides gene therapeutic agents to prevent or reduce the accumulation of lipofuscin in patients, thereby providing a new clinical strategy for treating AMD.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B demonstrate cycloretinal catabolism with MsP1. FIG. 1A shows Time 0, in which cycloretinal is seen as yellow. FIG. 1B shows the cultures after 2 hours of incubation, in which clearance of cycloretinal was observed.

FIG. 2 shows estimation of cycloretinal degradation products produced per unit of time.

FIG. 3 shows cycloretinal enzymatic degradation fragments observed by LC-MSMS.

FIG. 4 displays the fragmentation pathway for the degradation of cycloretinal in the presence of MsP1.

FIG. 5 shows the mechanism for the activity of MsP1 in the presence of compound B, where Cpd I is the iron (IV) oxo⁺⁻ intermediate responsible for peroxidase activity.

FIG. 6A shows cytotoxicity comparison of 2,4-DMB to that of cycloretinal within ARPE-19 cells for substrates delivered to cells in the absence of light. FIG. 6B shows cytotoxicity comparison of 2,4-DMB to that of cycloretinal within ARPE-19 cells for substrates delivered to cells in the presence of 430 nm light.

FIGS. 7A-7D show ARPE-19 cytotoxicity assays for cycloretinal MsP1 degradation products β-Ionone and β-cyclocitral. FIGS. 7A and 7C display substrates delivered to cells in the absence of light. FIGS. 7B and 7D display substrates delivered to cells in the presence of light (430 nm).

FIGS. 8A-8D show ARPE-19 cytotoxicity assays for cycloretinal MsP1 degradation products glyoxal and methylglyoxal. FIGS. 8A and 8C display substrates delivered to cells in the absence of light. FIGS. 8B and 8D display substrates delivered to cells in the presence of light (430 nm).

FIG. 9 shows a comparison of enzyme kinetics of wild-type MsP1 and select active site residues.

FIG. 10 shows MsP1 heme porphyrin activation.

FIGS. 11A-11B display the effect of pH on MsP1 catalyzed degradation of cycloretinal.

### DETAILED DESCRIPTION

Various embodiments of the invention are described herein as follows. In an illustrative aspect, a pharmaceutical composition is provided. The pharmaceutical composition comprises i) a MsP1 enzyme and ii) one or more pharmaceutically acceptable carriers. An MsP1 is known to the skilled artisan as a peroxidase enzyme from the organism Marasmius scorodonius.

In an embodiment, the MsP1 enzyme comprises an amino acid sequence encoding an MsP1 protein. In an embodiment, the amino acid sequence comprises SEQ ID NO: 1.

(513 amino acids):
SEQ ID NO: 1
MKLFSASVFAAIIASHYASATAHIRAPNVKPRRTNSLLTAPPQQPPLPS
AQQAASASSSAGLNLTDIQGDILIGMKKNKELFFFFSITDAATFKAKLG
SDILELITSTNQLLAVATQPITAVNVAFSSTGLKALGITDDLKDPVFEA
GMLSNAVSDLSDPGTGNWVPGFVGTSVHGVFLLASDTIDNVNTELANIQ
TILNGSITEIHRLQGEARPGDQQGHEHFGFMDGISNPAVDGFTPPAEIR
PGQALIPPGIMLLGEANDTFQNDRPPWAKDGSFLVFRQMQQRAPEFNKF
LQDHALNMPNMTSEQGADLLGARIVGRWKSDAPIDLTPLVDDPVLAADN
QRNNNFDFSDATNQTRCPFSAHIRKANPRGDLGGINKFPNQHIIRAGIP
YGPEVTDAEKASNSSSTDPSLERGLAFVAYQSNIQNGFVFLQKNWVDNT
NFFRPGTGVDPLIGTNSRNSGTDAPNTPRVVSGLDPNNATSTIEIGIDF
VVSRGGEYFFSPSLSAIRTVLSV

In an embodiment, the amino acid sequence comprises at least 80% sequence identity to the sequence of SEQ ID NO: 1. In an embodiment, the amino acid sequence comprises at least 85% sequence identity to the sequence of SEQ ID NO: 1. In an embodiment, the amino acid sequence comprises at least 90% sequence identity to the sequence of SEQ ID NO: 1. In an embodiment, the amino acid sequence comprises at least 95% sequence identity to the sequence of SEQ ID NO: 1. In an embodiment, the amino acid sequence comprises at least 96% sequence identity to the sequence of SEQ ID NO: 1. In an embodiment, the amino acid sequence comprises at least 97% sequence identity to the sequence of SEQ ID NO: 1. In an embodiment, the amino acid sequence comprises at least 98% sequence identity to the sequence of SEQ ID NO: 1. In an embodiment, the amino acid sequence comprises at least 99% sequence identity to the sequence of SEQ ID NO: 1.

In an embodiment, the amino acid sequence consists of at least 80% sequence identity to the sequence of SEQ ID NO: 1. In an embodiment, the amino acid sequence consists of at least 85% sequence identity to the sequence of SEQ ID NO: 1. In an embodiment, the amino acid sequence consists of at least 90% sequence identity to the sequence of SEQ ID NO: 1. In an embodiment, the amino acid sequence consists of at least 95% sequence identity to the sequence of SEQ ID NO: 1. In an embodiment, the amino acid sequence consists of at least 96% sequence identity to the sequence of SEQ ID NO: 1. In an embodiment, the amino acid sequence consists of at least 97% sequence identity to the sequence of SEQ ID NO: 1. In an embodiment, the amino acid sequence consists of at least 98% sequence identity to the sequence of SEQ ID NO: 1. In an embodiment, the amino acid sequence consists of at least 99% sequence identity to the sequence of SEQ ID NO: 1.

In an embodiment, the amino acid sequence comprises an aspartate (D) amino acid at position 228 of SEQ ID NO: 1. In an embodiment, the amino acid sequence comprises a histidine (H) amino acid at position 365 of SEQ ID NO: 1. In an embodiment, the amino acid sequence comprises an arginine (R) amino acid at position 388 of SEQ ID NO: 1.

In an embodiment, the MsP1 enzyme comprises a nucleic acid sequence encoding an MsP1 protein. In an embodiment, the nucleic acid sequence comprises SEQ ID NO: 2.

(2093 nucleotides):
SEQ ID NO: 2
atgaagcttttttctgcctccgtttttgctgctatcatcgctagtcacta
tgcgtcagcgactgcccacatcagggctcccaacgtgaagccaaggagga
caaactcacttctgactagtgagagcgttccactcctttccaaagtagtt
gcttatcggggtttcgaaattctctagctccccctcaacagcctccgctt
ccatctgctcaacaggctgcaagtgcctctagcagtgctggcttgaatct
caccgacatccagggtgatattctgtacgtatctttcgatatttcttgtg
tttatcgcagctaatgtacctgtttcaggatcggcatgaagaagaacaag
gaactatttttcttcttcagcatcaccgacgccgctactttcaaggtatt
tgcttttatatctgtatttcgaacatattttcacagttaatttcaaggct
aagctaggatccgacattcttgaactaatcacatcgaccaatcagttact
cgccgtcgccactcagcctatcacggctgtcaacgtcgctttctctagca
ctggcctcaaggcattgggtatcacagatgatctgaaggatcctgtcttc
gaggccggaatgctcagcaacgcagtgagcgacttgagcgatccagggac
cggcaattgggtgcctgggtttgtcggcaccagtgttcatggcgttttcc
tacttgcatcggacaccattgacaatgtaaacaccgagctggccaacatc
caaaccattttgaatggctcgatcacggagattcatcgtttgcaagggga
ggctcgacccggtgaccagcaaggtcacgaacgtaagatatttgtttccc
gctccgtactctgctcatctgcatatcgacagactttggattcatggatg
gaatcagtaacccggccgttgatggatttacacctccagcggaaataaga
cctggacaagctttaattccgcctggtatcatgcttctcggagaggcaaa
cgacacttttcagaatgatcgtcctccgtgggccaaagatggttccttcc
ttgtcttccgtcaaatgcaacagcgcgcgcccgagttcaacaagttcctg
caagatcacgctcttaacatgccgaatatgacatccgagcaaggcgctga
tctccttggtgccaggattgtaggacgatggaaaagtgtaagctctctct
tcgtatagatggcatcctactcagctcaacgttcggtggttaggatgctc
ctattgacctcactccgttggtcgatgacccagtgttggctgctgacaat
cagcgaaataacaacttcgacttttctgacgccacgaatcagacacgttg
ccctttctctgctcatatccgcaaggctaacccgcgcggtgatcttgggg
gtattaataaattcccaaaccaacacataatccgagcgggaattccgtat
ggacccgaaggtgccatttattctgcatcgtccctgcagcataattctga
ttccttctttttggttcagttaccgacgctgaaaaagcgtcaaatagctc
tagcactgaccctagtctggagcgtggtctggcgtttggtgagtagaaag
ccctatttagagcgacaggcctaatttggaagattctagtggcctatcag
tctaatatccagaacggattcgtattccttcaaaagaattgggttgataa
tacgaagtgcgttctgccgaattctgtcctgcagcttgaaactaatcaca
ttgtaaaaacgcagtttcttccgacccggcactggtgtagatcctctcat
cggtaatgcagctgcattcgaagaaagccctcctagtcgttgctaatgaa
tacgtttttaggtacaaattctcgtaacagtggcaccgatgcccccaaca
cgcctcgtgtcgtcagcggcttggatcctaataacgctacgagcaccatc
gaaattggtatcggtatgtgtatccgcttcctatgaaacttacattcccg
ctaaccctgtgctcagatttcgtagtttctcgtggaggagaatacttctt
ctcgccctcactttctgcgatcaggactgtgctttcagtctag

In an embodiment, the nucleic acid sequence comprises at least 80% sequence identity to the sequence of SEQ ID NO: 2. In an embodiment, the nucleic acid sequence comprises at least 85% sequence identity to the sequence of SEQ ID NO: 2. In an embodiment, the nucleic acid sequence comprises at least 90% sequence identity to the sequence of SEQ ID NO: 2. In an embodiment, the nucleic acid sequence comprises at least 95% sequence identity to the sequence of SEQ ID NO: 2. In an embodiment, the nucleic acid sequence comprises at least 96% sequence identity to the sequence of SEQ ID NO: 2. In an embodiment, the nucleic acid sequence comprises at least 97% sequence identity to the sequence of SEQ ID NO: 2. In an embodiment, the nucleic acid sequence comprises at least 98% sequence identity to the sequence of SEQ ID NO: 2. In an embodiment, the nucleic acid sequence comprises at least 99% sequence identity to the sequence of SEQ ID NO: 2.

In an embodiment, the nucleic acid sequence consists of at least 80% sequence identity to the sequence of SEQ ID NO: 2. In an embodiment, the nucleic acid sequence consists of at least 85% sequence identity to the sequence of SEQ ID NO: 2. In an embodiment, the nucleic acid sequence consists of at least 90% sequence identity to the sequence of SEQ ID NO: 2. In an embodiment, the nucleic acid sequence consists of at least 95% sequence identity to the sequence of SEQ ID NO: 2. In an embodiment, the nucleic acid sequence consists of at least 96% sequence identity to the sequence of SEQ ID NO: 2. In an embodiment, the nucleic acid sequence consists of at least 97% sequence identity to the sequence of SEQ ID NO: 2. In an embodiment, the nucleic acid sequence consists of at least 98% sequence identity to the sequence of SEQ ID NO: 2. In an embodiment, the nucleic acid sequence consists of at least 99% sequence identity to the sequence of SEQ ID NO: 2.

In an embodiment, the nucleic acid sequence provides for an aspartate (D) amino acid at position 228 of the amino acid sequence of SEQ ID NO: 1. In an embodiment, the nucleic acid sequence provides for a histidine (H) amino acid at position 365 of the amino acid sequence of SEQ ID NO: 1. In an embodiment, the nucleic acid sequence provides for an arginine (R) amino acid at position 388 of the amino acid sequence of SEQ ID NO: 1.

In an embodiment, the nucleic acid sequence comprises SEQ ID NO: 3.

(1539 nucleotides):
SEQ ID NO: 3
atgaagcttttttctgcctccgtttttgctgctatcatcgctagtcacta
tgcgtcagcgactgcccacatcagggctcccaacgtgaagccaaggagga
caaactcacttctgactgctccccctcaacagcctccgcttccatctgct
caacaggctgcaagtgcctctagcagtgctggcttgaatctcaccgacat
ccagggtgatattctgatcggcatgaagaagaacaaggaactatttttct
tcttcagcatcaccgacgccgctactttcaaggctaagctaggatccgac
attcttgaactaatcacatcgaccaatcagttactcgccgtcgccactca
gcctatcacggctgtcaacgtcgctttctctagcactggcctcaaggcat
tgggtatcacagatgatctgaaggatcctgtcttcgaggccggaatgctc
agcaacgcagtgagcgacttgagcgatccagggaccggcaattgggtgcc
tgggtttgtcggcaccagtgttcatggcgttttcctacttgcatcggaca
ccattgacaatgtaaacaccgagctggccaacatccaaaccattttgaat
ggctcgatcacggagattcatcgtttgcaaggggaggctcgacccggtga
ccagcaaggtcacgaacactttggattcatggatggaatcagtaacccgg
ccgttgatggatttacacctccagcggaaataagacctggacaagcttta
attccgcctggtatcatgcttctcggagaggcaaacgacacttttcagaa
tgatcgtcctccgtgggccaaagatggttccttccttgtcttccgtcaaa
tgcaacagcgcgcgcccgagttcaacaagttcctgcaagatcacgctctt
aacatgccgaatatgacatccgagcaaggcgctgatctccttggtgccag
gattgtaggacgatggaaaagtgatgctcctattgacctcactccgttgg
tcgatgacccagtgttggctgctgacaatcagcgaaataacaacttcgac
ttttctgacgccacgaatcagacacgttgccctttctctgctcatatccg
caaggctaacccgcgcggtgatcttgggggtattaataaattcccaaacc
aacacataatccgagcgggaattccgtatggacccgaagttaccgacgct
gaaaaagcgtcaaatagctctagcactgaccctagtctggagcgtggtct
ggcgtttgtggcctatcagtctaatatccagaacggattcgtattccttc
aaaagaattgggttgataatacgaatttcttccgacccggcactggtgta
gatcctctcatcggtacaaattctcgtaacagtggcaccgatgcccccaa
cacgcctcgtgtcgtcagcggcttggatcctaataacgctacgagcacca
tcgaaattggtatcgatttcgtagtttctcgtggaggagaatacttcttc
tcgccctcactttctgcgatcaggactgtgctttcagtc

In an embodiment, the nucleic acid sequence comprises at least 80% sequence identity to the sequence of SEQ ID NO: 3. In an embodiment, the nucleic acid sequence comprises at least 85% sequence identity to the sequence of SEQ ID NO: 3. In an embodiment, the nucleic acid sequence comprises at least 90% sequence identity to the sequence of SEQ ID NO: 3. In an embodiment, the nucleic acid sequence comprises at least 95% sequence identity to the sequence of SEQ ID NO: 3. In an embodiment, the nucleic acid sequence comprises at least 96% sequence identity to the sequence of SEQ ID NO: 3. In an embodiment, the nucleic acid sequence comprises at least 97% sequence identity to the sequence of SEQ ID NO: 3. In an embodiment, the nucleic acid sequence comprises at least 98% sequence identity to the sequence of SEQ ID NO: 3. In an embodiment, the nucleic acid sequence comprises at least 99% sequence identity to the sequence of SEQ ID NO: 3.

In an embodiment, the nucleic acid sequence consists of at least 80% sequence identity to the sequence of SEQ ID NO: 3. In an embodiment, the nucleic acid sequence consists of at least 85% sequence identity to the sequence of SEQ ID NO: 3. In an embodiment, the nucleic acid sequence consists of at least 90% sequence identity to the sequence of SEQ ID NO: 3. In an embodiment, the nucleic acid sequence consists of at least 95% sequence identity to the sequence of SEQ ID NO: 3. In an embodiment, the nucleic acid sequence consists of at least 96% sequence identity to the sequence of SEQ ID NO: 3. In an embodiment, the nucleic acid sequence consists of at least 97% sequence identity to the sequence of SEQ ID NO: 3. In an embodiment, the nucleic acid sequence consists of at least 98% sequence identity to the sequence of SEQ ID NO: 3. In an embodiment, the nucleic acid sequence consists of at least 99% sequence identity to the sequence of SEQ ID NO: 3.

In an embodiment, the nucleic acid sequence provides for an aspartate (D) amino acid at position 228 of the amino acid sequence of SEQ ID NO: 1. In an embodiment, the nucleic acid sequence provides for a histidine (H) amino acid at position 365 of the amino acid sequence of SEQ ID NO: 1. In an embodiment, the nucleic acid sequence provides for an arginine (R) amino acid at position 388 of the amino acid sequence of SEQ ID NO: 1.

In an embodiment, the nucleic acid sequence is a native sequence that has been modified to comprise codons optimized for expression in prokaryotic cells. In an embodiment, the nucleic acid sequence comprises SEQ ID NO: 4.

(1539 nucleotides):
SEQ ID NO: 4
atgaaactgttctccgcatcggtctttgcagccatcatcgcttctcacta
cgcctcagccacggcacacatccgcgctccgaatgttaaaccgcgtcgca
ccaacagcctgctgacggcaccgccgcagcaaccgccgctgccgagcgca
cagcaagcggcctctgcgagctctagtgccggtctgaatctgaccgatat
ccagggcgacattctgatcggtatgaagaaaaacaaagaactgtttttct
ttttctcaattaccgatgcagctacgtttaaagcgaaactgggcagtgac
atcctggaactgattaccagcaccaatcagctgctggcagtggctaccca
accgatcacggcagttaacgtcgctttttcctcaaccggcctgaaagccc
tgggtattacggatgacctgaaagatccggtcttcgaagcgggtatgctg
agcaatgccgtgagtgatctgtccgacccgggcaccggtaactgggtccc
gggctttgtgggtacgagcgtgcatggcgttttcctgctggcgtctgata
ccattgacaatgtgaacacggaactggccaatatccaaaccattctgaac
ggtagtatcacggaaattcaccgtctgcagggtgaagcacgtccgggtga
tcagcaaggtcatgaacactttggcttcatggatggtatctccaacccgg
cagttgacggttttaccccgccggcagaaattcgtccgggtcaggctctg
atcccgccgggtattatgctgctgggtgaagcaaatgataccttccagaa
cgaccgcccgccgtgggctaaagatggtagctttctggtgttccgtcaga
tgcagcaacgcgcaccggaatttaataaattcctgcaagatcatgctctg
aatatgccgaacatgaccagcgaacagggtgcggatctgctgggtgcacg
tatcgttggccgctggaaatctgatgcgccgattgacctgaccccgctgg
ttgatgacccggtcctggcagccgataaccaacgtaacaacaacttcgat
ttctcagacgccaccaaccagacgcgctgcccgttttcggcacatatccg
taaagctaatccgcgcggcgatctgggcggtattaataaattcccgaacc
agcacattatccgtgcgggcattccgtatggtccggaagttaccgatgcg
gaaaaagcctcaaattcgagctctacggacccgtcgctggaacgcggtct
ggcatttgtcgcttaccagagtaatatccaaaacggctttgtgttcctgc
agaaaaactgggttgataataccaactttttccgtccgggcacgggtgtt
gacccgctgattggcaccaacagccgtaacagcggcaccgatgcaccgaa
tacgccgcgcgtggttagcggtctggacccgaataacgcgaccagcacca
ttgaaatcggcattgattttgtcgtgtctcgtggcggcgaatacttcttc
tccccgtccctgtcggctatccgtaccgtcctgtccgtt

In an embodiment, the nucleic acid sequence comprises at least 80% sequence identity to the sequence of SEQ ID NO: 4. In an embodiment, the nucleic acid sequence comprises at least 85% sequence identity to the sequence of SEQ ID NO: 4. In an embodiment, the nucleic acid sequence comprises at least 90% sequence identity to the sequence of SEQ ID NO: 4. In an embodiment, the nucleic acid sequence comprises at least 95% sequence identity to the sequence of SEQ ID NO: 4. In an embodiment, the nucleic acid sequence comprises at least 96% sequence identity to the sequence of SEQ ID NO: 4. In an embodiment, the nucleic acid sequence comprises at least 97% sequence identity to the sequence of SEQ ID NO: 4. In an embodiment, the nucleic acid sequence comprises at least 98% sequence identity to the sequence of SEQ ID NO: 4. In an embodiment, the nucleic acid sequence comprises at least 99% sequence identity to the sequence of SEQ ID NO: 4.

In an embodiment, the nucleic acid sequence consists of at least 80% sequence identity to the sequence of SEQ ID NO: 4. In an embodiment, the nucleic acid sequence consists of at least 85% sequence identity to the sequence of SEQ ID NO: 4. In an embodiment, the nucleic acid sequence consists of at least 90% sequence identity to the sequence of SEQ ID NO: 4. In an embodiment, the nucleic acid sequence consists of at least 95% sequence identity to the sequence of SEQ ID NO: 4. In an embodiment, the nucleic acid sequence consists of at least 96% sequence identity to the sequence of SEQ ID NO: 4. In an embodiment, the nucleic acid sequence consists of at least 97% sequence identity to the sequence of SEQ ID NO: 4. In an embodiment, the nucleic acid sequence consists of at least 98% sequence identity to the sequence of SEQ ID NO: 4. In an embodiment, the nucleic acid sequence consists of at least 99% sequence identity to the sequence of SEQ ID NO: 4.

In an embodiment, the nucleic acid sequence provides for an aspartate (D) amino acid at position 228 of the amino acid sequence of SEQ ID NO: 1. In an embodiment, the nucleic acid sequence provides for a histidine (H) amino acid at position 365 of the amino acid sequence of SEQ ID NO: 1. In an embodiment, the nucleic acid sequence provides for an arginine (R) amino acid at position 388 of the amino acid sequence of SEQ ID NO: 1.

In an embodiment, the pharmaceutical composition is an oral formulation. In an embodiment, the oral formulation is selected from the group consisting of a tablet, a capsule, a suspension, an emulsion, a syrup, a colloidal dispersion, a dispersion, and an effervescent composition. In an embodiment, the oral formulation is a suspension. In an embodiment, the oral formulation is a reconstitutable suspension.

In an embodiment, the pharmaceutical composition is a parenteral formulation. In an embodiment, the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

In an embodiment, the pharmaceutical composition further comprises a second therapeutic agent. In an embodiment, the pharmaceutical composition is formulated as a single dose. In an embodiment, the pharmaceutical composition is formulated as a single unit dose.

In an illustrative aspect, a method of treating an eye disorder in a patient in need thereof is provided. The method comprises the step of administering a therapeutically effective amount of a pharmaceutical composition to the patient, In an embodiment, the pharmaceutical composition comprises i) a MsP1 enzyme and ii) one or more pharmaceutically acceptable carriers.

The previously described embodiments of the pharmaceutical composition are applicable to the method of treating an eye disorder in a patient described herein.

In an embodiment, the patient is an animal. In an embodiment, the animal is a mammal. In an embodiment, the animal is a human.

In an embodiment, the eye disorder is age-related macular degeneration (AMD). In an embodiment, the AMD is dry form AMD. In an embodiment, the AMD is wet form AMD.

In an embodiment, the administration provides a reduction in one or more symptoms of the eye disorder in the patient. In an embodiment, the one or more symptoms comprise presence of drusen. In an embodiment, the one or more symptoms comprise blurred vision. In an embodiment, the one or more symptoms comprise distorted vision. In an embodiment, the one or more symptoms comprise color perception.

In an embodiment, the administration degrades cycloretinal in the patient. In an embodiment, the cycloretinal is degraded to beta-ionone. In an embodiment, the cycloretinal is degraded to 2,4-dimethylbenzaldehyde (2,4-DMB). In an embodiment, the administration provides a reduction in clinical progression of the eye disorder in the patient.

In an embodiment, the pharmaceutical composition is administered to the patient at a dose of about 0.001 to about 1000 mg of the MsP1 enzyme per kg of body weight. In an embodiment, the pharmaceutical composition is administered to the patient at a dose of about 0.001 to about 100 mg of the MsP1 enzyme per kg of body weight. In an embodiment, the pharmaceutical composition is administered to the patient at a dose of about 0.001 to about 10 mg of the MsP1 enzyme per kg of body weight. In an embodiment, the pharmaceutical composition is administered to the patient at a dose of about 1 to about 5 mg of the MsP1 enzyme per kg of body weight. In an embodiment, the pharmaceutical composition is administered to the patient at a dose of about 1 mg of the MsP1 enzyme per kg of body weight. In an embodiment, the pharmaceutical composition is administered to the patient at a dose of about 2 mg of the MsP1 enzyme per kg of body weight. In an embodiment, the pharmaceutical composition is administered to the patient at a dose of about 3 mg of the MsP1 enzyme per kg of body weight. In an embodiment, the pharmaceutical composition is administered to the patient at a dose of about 4 mg of the MsP1 enzyme per kg of body weight. In an embodiment, the pharmaceutical composition is administered to the patient at a dose of about 5 mg of the MsP1 enzyme per kg of body weight.

In an embodiment, the administration is an oral administration. In an embodiment, the oral administration is selected from the group consisting of a tablet, a capsule, a suspension, an emulsion, a syrup, a colloidal dispersion, a dispersion, and an effervescent composition. In an embodiment, the oral formulation is a suspension. In an embodiment, the oral formulation is a reconstitutable suspension.

In an embodiment, the administration is a parenteral administration. In an embodiment, the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

In an embodiment, the pharmaceutical composition is administered as a single dose. In an embodiment, the pharmaceutical composition is administered as a single unit dose.

In an embodiment, the method further comprises administration of a second therapeutic agent to the patient. In an embodiment, the second therapeutic agent is lutein. In an embodiment, the second therapeutic agent is zeaxanthin. In an embodiment, the second therapeutic agent is zinc. In an embodiment, the second therapeutic agent is Vitamin C. In an embodiment, the second therapeutic agent is Vitamin E.

In an illustrative aspect, a method of reducing cycloretinal in a patient in need thereof is provided. The method comprises the step of administering a therapeutically effective amount of a pharmaceutical composition to the patient, In an embodiment, the pharmaceutical composition comprises i) a MsP1 enzyme or a variant thereof and ii) one or more pharmaceutically acceptable carriers.

The previously described embodiments of the pharmaceutical composition are applicable to the method of reducing cycloretinal in a patient described herein.

In an embodiment, the cycloretinal is reduced in an eye of the patient. In an embodiment, the eye comprises a retina, and wherein the cycloretinal is reduced in the retina. In an embodiment, the retina comprises a retinal pigment epithelium, and wherein the cycloretinal is reduced in the retinal pigment epithelium.

In an embodiment, the administration degrades cycloretinal in the patient. In an embodiment, the cycloretinal is degraded to beta-ionone. In an embodiment, the cycloretinal is degraded to 2,4-dimethylbenzaldehyde (2,4-DMB). In an embodiment, the administration provides a reduction in clinical progression of the eye disorder in the patient.

In an embodiment, the pharmaceutical composition is administered to the patient at a dose of about 0.001 to about 1000 mg of the MsP1 enzyme per kg of body weight. In an embodiment, the pharmaceutical composition is administered to the patient at a dose of about 0.001 to about 100 mg of the MsP1 enzyme per kg of body weight. In an embodiment, the pharmaceutical composition is administered to the patient at a dose of about 0.001 to about 10 mg of the MsP1 enzyme per kg of body weight. In an embodiment, the pharmaceutical composition is administered to the patient at a dose of about 1 to about 5 mg of the MsP1 enzyme per kg of body weight. In an embodiment, the pharmaceutical composition is administered to the patient at a dose of about 1 mg of the MsP1 enzyme per kg of body weight. In an embodiment, the pharmaceutical composition is administered to the patient at a dose of about 2 mg of the MsP1 enzyme per kg of body weight. In an embodiment, the pharmaceutical composition is administered to the patient at a dose of about 3 mg of the MsP1 enzyme per kg of body weight. In an embodiment, the pharmaceutical composition is administered to the patient at a dose of about 4 mg of the MsP1 enzyme per kg of body weight. In an embodiment, the pharmaceutical composition is administered to the patient at a dose of about 5 mg of the MsP1 enzyme per kg of body weight.

In an embodiment, the administration is an oral administration. In an embodiment, the oral administration is selected from the group consisting of a tablet, a capsule, a suspension, an emulsion, a syrup, a colloidal dispersion, a dispersion, and an effervescent composition. In an embodiment, the oral formulation is a suspension. In an embodiment, the oral formulation is a reconstitutable suspension.

In an embodiment, the administration is a parenteral administration. In an embodiment, the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

In an embodiment, the pharmaceutical composition is administered as a single dose. In an embodiment, the pharmaceutical composition is administered as a single unit dose.

In an embodiment, the method further comprises administration of a second therapeutic agent to the patient. In an embodiment, the second therapeutic agent is lutein. In an embodiment, the second therapeutic agent is zeaxanthin. In an embodiment, the second therapeutic agent is zinc. In an embodiment, the second therapeutic agent is Vitamin C. In an embodiment, the second therapeutic agent is Vitamin E.

In an illustrative aspect, a recombinant polynucleotide is provided. The recombinant polynucleotide comprises a heterologous regulatory element operably linked to a nucleic acid sequence encoding a MsP1 protein.

In an embodiment, the MsP1 protein comprises SEQ ID NO: 1. In an embodiment, the MsP1 protein comprises at least 80% sequence identity to the sequence of SEQ ID NO: 1. In an embodiment, the MsP1 protein comprises at least 85% sequence identity to the sequence of SEQ ID NO: 1. In an embodiment, the MsP1 protein comprises at least 90% sequence identity to the sequence of SEQ ID NO: 1. In an embodiment, the MsP1 protein comprises at least 95% sequence identity to the sequence of SEQ ID NO: 1. In an embodiment, the MsP1 protein comprises at least 96% sequence identity to the sequence of SEQ ID NO: 1. In an embodiment, the MsP1 protein comprises at least 97% sequence identity to the sequence of SEQ ID NO: 1. In an embodiment, the MsP1 protein comprises at least 98% sequence identity to the sequence of SEQ ID NO: 1. In an embodiment, the MsP1 protein comprises at least 99% sequence identity to the sequence of SEQ ID NO: 1.

In an embodiment, the MsP1 protein consists of at least 80% sequence identity to the sequence of SEQ ID NO: 1. In an embodiment, the MsP1 protein consists of at least 85% sequence identity to the sequence of SEQ ID NO: 1. In an embodiment, the MsP1 protein consists of at least 90% sequence identity to the sequence of SEQ ID NO: 1. In an embodiment, the MsP1 protein consists of at least 95% sequence identity to the sequence of SEQ ID NO: 1. In an embodiment, the MsP1 protein consists of at least 96% sequence identity to the sequence of SEQ ID NO: 1. In an embodiment, the MsP1 protein consists of at least 97% sequence identity to the sequence of SEQ ID NO: 1. In an embodiment, the MsP1 protein consists of at least 98% sequence identity to the sequence of SEQ ID NO: 1. In an embodiment, the MsP1 protein consists of at least 99% sequence identity to the sequence of SEQ ID NO: 1.

In an embodiment, the heterologous regulatory element is a promoter that functions in prokaryotic cells.

In an embodiment, the nucleic acid sequence comprises SEQ ID NO: 2. In an embodiment, the nucleic acid sequence comprises at least 80% sequence identity to the sequence of SEQ ID NO: 2. In an embodiment, the nucleic acid sequence comprises at least 85% sequence identity to the sequence of SEQ ID NO: 2. In an embodiment, the nucleic acid sequence comprises at least 90% sequence identity to the sequence of SEQ ID NO: 2. In an embodiment, the nucleic acid sequence comprises at least 95% sequence identity to the sequence of SEQ ID NO: 2. In an embodiment, the nucleic acid sequence comprises at least 96% sequence identity to the sequence of SEQ ID NO: 2. In an embodiment, the nucleic acid sequence comprises at least 97% sequence identity to the sequence of SEQ ID NO: 2. In an embodiment, the nucleic acid sequence comprises at least 98% sequence identity to the sequence of SEQ ID NO: 2. In an embodiment, the nucleic acid sequence comprises at least 99% sequence identity to the sequence of SEQ ID NO: 2.

In an embodiment, the nucleic acid sequence consists of at least 80% sequence identity to the sequence of SEQ ID NO: 2. In an embodiment, the nucleic acid sequence consists of at least 85% sequence identity to the sequence of SEQ ID NO: 2. In an embodiment, the nucleic acid sequence consists of at least 90% sequence identity to the sequence of SEQ ID NO: 2. In an embodiment, the nucleic acid sequence consists of at least 95% sequence identity to the sequence of SEQ ID NO: 2. In an embodiment, the nucleic acid sequence consists of at least 96% sequence identity to the sequence of SEQ ID NO: 2. In an embodiment, the nucleic acid sequence consists of at least 97% sequence identity to the sequence of SEQ ID NO: 2. In an embodiment, the nucleic acid sequence consists of at least 98% sequence identity to the sequence of SEQ ID NO: 2. In an embodiment, the nucleic acid sequence consists of at least 99% sequence identity to the sequence of SEQ ID NO: 2.

In an embodiment, the nucleic acid sequence comprises SEQ ID NO: 3. In an embodiment, the nucleic acid sequence comprises at least 80% sequence identity to the sequence of SEQ ID NO: 3. In an embodiment, the nucleic acid sequence comprises at least 85% sequence identity to the sequence of SEQ ID NO: 3. In an embodiment, the nucleic acid sequence comprises at least 90% sequence identity to the sequence of SEQ ID NO: 3. In an embodiment, the nucleic acid sequence comprises at least 95% sequence identity to the sequence of SEQ ID NO: 3. In an embodiment, the nucleic acid sequence comprises at least 96% sequence identity to the sequence of SEQ ID NO: 3. In an embodiment, the nucleic acid sequence comprises at least 97% sequence identity to the sequence of SEQ ID NO: 3. In an embodiment, the nucleic acid sequence comprises at least 98% sequence identity to the sequence of SEQ ID NO: 3. In an embodiment, the nucleic acid sequence comprises at least 99% sequence identity to the sequence of SEQ ID NO: 3.

In an embodiment, the nucleic acid sequence consists of at least 80% sequence identity to the sequence of SEQ ID NO: 3. In an embodiment, the nucleic acid sequence consists of at least 85% sequence identity to the sequence of SEQ ID NO: 3. In an embodiment, the nucleic acid sequence consists of at least 90% sequence identity to the sequence of SEQ ID NO: 3. In an embodiment, the nucleic acid sequence consists of at least 95% sequence identity to the sequence of SEQ ID NO: 3. In an embodiment, the nucleic acid sequence consists of at least 96% sequence identity to the sequence of SEQ ID NO: 3. In an embodiment, the nucleic acid sequence consists of at least 97% sequence identity to the sequence of SEQ ID NO: 3. In an embodiment, the nucleic acid sequence consists of at least 98% sequence identity to the sequence of SEQ ID NO: 3. In an embodiment, the nucleic acid sequence consists of at least 99% sequence identity to the sequence of SEQ ID NO: 3.

In an embodiment, the nucleic acid sequence is a native sequence that has been modified to comprise codons optimized for expression in prokaryotic cells. In an embodiment, the nucleic acid sequence comprises SEQ ID NO: 4.

In an embodiment, the nucleic acid sequence comprises at least 80% sequence identity to the sequence of SEQ ID NO: 4. In an embodiment, the nucleic acid sequence comprises at least 85% sequence identity to the sequence of SEQ ID NO: 4. In an embodiment, the nucleic acid sequence comprises at least 90% sequence identity to the sequence of SEQ ID NO: 4. In an embodiment, the nucleic acid sequence comprises at least 95% sequence identity to the sequence of SEQ ID NO: 4. In an embodiment, the nucleic acid sequence comprises at least 96% sequence identity to the sequence of SEQ ID NO: 4. In an embodiment, the nucleic acid sequence comprises at least 97% sequence identity to the sequence of SEQ ID NO: 4. In an embodiment, the nucleic acid sequence comprises at least 98% sequence identity to the sequence of SEQ ID NO: 4. In an embodiment, the nucleic acid sequence comprises at least 99% sequence identity to the sequence of SEQ ID NO: 4.

In an embodiment, the nucleic acid sequence consists of at least 80% sequence identity to the sequence of SEQ ID NO: 4. In an embodiment, the nucleic acid sequence consists of at least 85% sequence identity to the sequence of SEQ ID NO: 4. In an embodiment, the nucleic acid sequence consists of at least 90% sequence identity to the sequence of SEQ ID NO: 4. In an embodiment, the nucleic acid sequence consists of at least 95% sequence identity to the sequence of SEQ ID NO: 4. In an embodiment, the nucleic acid sequence consists of at least 96% sequence identity to the sequence of SEQ ID NO: 4. In an embodiment, the nucleic acid sequence consists of at least 97% sequence identity to the sequence of SEQ ID NO: 4. In an embodiment, the nucleic acid sequence consists of at least 98% sequence identity to the sequence of SEQ ID NO: 4. In an embodiment, the nucleic acid sequence consists of at least 99% sequence identity to the sequence of SEQ ID NO: 4.

In an illustrative aspect, an expression vector is provided. The expression vector comprises a recombinant polynucleotide of any one of the above claims and a selectable marker gene.

In an embodiment, the selectable marker gene encodes for antibiotic resistance. In an embodiment, the selectable marker gene is neomycin (Neo). In an embodiment, the selectable marker gene is bleomycin (Ble). In an embodiment, the selectable marker gene is blasticidin (Bsr/Bsd). In an embodiment, the selectable marker gene confers neomycin resistance. In an embodiment, the selectable marker gene confers bleomycin resistance. In an embodiment, the selectable marker gene confers blasticidin resistance.

All texts, figures, tables, supplemental information, and the like associated with the journal article of Perveen et al., “Catabolism of the Lipofuscin Cycloretinal by MsP1,” Biochemistry, 2022 Nov. 15; 61(22):2560-2567, are incorporated herein by reference in their entirety.

The following numbered embodiments are contemplated and are non-limiting:

1. A pharmaceutical composition comprising i) a MsP1 enzyme and ii) one or more pharmaceutically acceptable carriers.

2. The pharmaceutical composition of clause 1, any other suitable clause, or any combination of suitable clauses, wherein the MsP1 enzyme comprises an amino acid sequence encoding an MsP1 protein.

3. The pharmaceutical composition of clause 2, any other suitable clause, or any combination of suitable clauses, wherein the amino acid sequence comprises SEQ ID NO: 1.

4. The pharmaceutical composition of clause 2, any other suitable clause, or any combination of suitable clauses, wherein the amino acid sequence comprises at least 80% sequence identity to the sequence of SEQ ID NO: 1.

5. The pharmaceutical composition of clause 2, any other suitable clause, or any combination of suitable clauses, wherein the amino acid sequence comprises at least 85% sequence identity to the sequence of SEQ ID NO: 1.

6. The pharmaceutical composition of clause 2, any other suitable clause, or any combination of suitable clauses, wherein the amino acid sequence comprises at least 90% sequence identity to the sequence of SEQ ID NO: 1.

7. The pharmaceutical composition of clause 2, any other suitable clause, or any combination of suitable clauses, wherein the amino acid sequence comprises at least 95% sequence identity to the sequence of SEQ ID NO: 1.

8. The pharmaceutical composition of clause 2, any other suitable clause, or any combination of suitable clauses, wherein the amino acid sequence comprises at least 96% sequence identity to the sequence of SEQ ID NO: 1.

9. The pharmaceutical composition of clause 2, any other suitable clause, or any combination of suitable clauses, wherein the amino acid sequence comprises at least 97% sequence identity to the sequence of SEQ ID NO: 1.

10. The pharmaceutical composition of clause 2, any other suitable clause, or any combination of suitable clauses, wherein the amino acid sequence comprises at least 98% sequence identity to the sequence of SEQ ID NO: 1.

11. The pharmaceutical composition of clause 2, any other suitable clause, or any combination of suitable clauses, wherein the amino acid sequence comprises at least 99% sequence identity to the sequence of SEQ ID NO: 1.

12. The pharmaceutical composition of clause 2, any other suitable clause, or any combination of suitable clauses, wherein the amino acid sequence consists of at least 80% sequence identity to the sequence of SEQ ID NO: 1.

13. The pharmaceutical composition of clause 2, any other suitable clause, or any combination of suitable clauses, wherein the amino acid sequence consists of at least 85% sequence identity to the sequence of SEQ ID NO: 1.

14. The pharmaceutical composition of clause 2, any other suitable clause, or any combination of suitable clauses, wherein the amino acid sequence consists of at least 90% sequence identity to the sequence of SEQ ID NO: 1.

15. The pharmaceutical composition of clause 2, any other suitable clause, or any combination of suitable clauses, wherein the amino acid sequence consists of at least 95% sequence identity to the sequence of SEQ ID NO: 1.

16. The pharmaceutical composition of clause 2, any other suitable clause, or any combination of suitable clauses, wherein the amino acid sequence consists of at least 96% sequence identity to the sequence of SEQ ID NO: 1.

17. The pharmaceutical composition of clause 2, any other suitable clause, or any combination of suitable clauses, wherein the amino acid sequence consists of at least 97% sequence identity to the sequence of SEQ ID NO: 1.

18. The pharmaceutical composition of clause 2, any other suitable clause, or any combination of suitable clauses, wherein the amino acid sequence consists of at least 98% sequence identity to the sequence of SEQ ID NO: 1.

19. The pharmaceutical composition of clause 2, any other suitable clause, or any combination of suitable clauses, wherein the amino acid sequence consists of at least 99% sequence identity to the sequence of SEQ ID NO: 1.

20. The pharmaceutical composition of clause 2, any other suitable clause, or any combination of suitable clauses, wherein the amino acid sequence comprises an aspartate (D) amino acid at position 228 of SEQ ID NO: 1.

21. The pharmaceutical composition of clause 2, any other suitable clause, or any combination of suitable clauses, wherein the amino acid sequence comprises a histidine (H) amino acid at position 365 of SEQ ID NO: 1.

22. The pharmaceutical composition of clause 2, any other suitable clause, or any combination of suitable clauses, wherein the amino acid sequence comprises an arginine (R) amino acid at position 388 of SEQ ID NO: 1.

23. The pharmaceutical composition of clause 1, any other suitable clause, or any combination of suitable clauses, wherein the MsP1 enzyme comprises a nucleic acid sequence encoding an MsP1 protein.

24. The pharmaceutical composition of clause 23, any other suitable clause, or any combination of suitable clauses, wherein the nucleic acid sequence comprises SEQ ID NO: 2.

25. The pharmaceutical composition of clause 23, any other suitable clause, or any combination of suitable clauses, wherein the nucleic acid sequence comprises at least 80% sequence identity to the sequence of SEQ ID NO: 2.

26. The pharmaceutical composition of clause 23, any other suitable clause, or any combination of suitable clauses, wherein the nucleic acid sequence comprises at least 85% sequence identity to the sequence of SEQ ID NO: 2.

27. The pharmaceutical composition of clause 23, any other suitable clause, or any combination of suitable clauses, wherein the nucleic acid sequence comprises at least 90% sequence identity to the sequence of SEQ ID NO: 2.

28. The pharmaceutical composition of clause 23, any other suitable clause, or any combination of suitable clauses, wherein the nucleic acid sequence comprises at least 95% sequence identity to the sequence of SEQ ID NO: 2.

29. The pharmaceutical composition of clause 23, any other suitable clause, or any combination of suitable clauses, wherein the nucleic acid sequence comprises at least 96% sequence identity to the sequence of SEQ ID NO: 2.

30. The pharmaceutical composition of clause 23, any other suitable clause, or any combination of suitable clauses, wherein the nucleic acid sequence comprises at least 97% sequence identity to the sequence of SEQ ID NO: 2.

31. The pharmaceutical composition of clause 23, any other suitable clause, or any combination of suitable clauses, wherein the nucleic acid sequence comprises at least 98% sequence identity to the sequence of SEQ ID NO: 2.

32. The pharmaceutical composition of clause 23, any other suitable clause, or any combination of suitable clauses, wherein the nucleic acid sequence comprises at least 99% sequence identity to the sequence of SEQ ID NO: 2.

33. The pharmaceutical composition of clause 23, any other suitable clause, or any combination of suitable clauses, wherein the nucleic acid sequence consists of at least 80% sequence identity to the sequence of SEQ ID NO: 2.

34. The pharmaceutical composition of clause 23, any other suitable clause, or any combination of suitable clauses, wherein the nucleic acid sequence consists of at least 85% sequence identity to the sequence of SEQ ID NO: 2.

35. The pharmaceutical composition of clause 23, any other suitable clause, or any combination of suitable clauses, wherein the nucleic acid sequence consists of at least 90% sequence identity to the sequence of SEQ ID NO: 2.

36. The pharmaceutical composition of clause 23, any other suitable clause, or any combination of suitable clauses, wherein the nucleic acid sequence consists of at least 95% sequence identity to the sequence of SEQ ID NO: 2.

37. The pharmaceutical composition of clause 23, any other suitable clause, or any combination of suitable clauses, wherein the nucleic acid sequence consists of at least 96% sequence identity to the sequence of SEQ ID NO: 2.

38. The pharmaceutical composition of clause 23, any other suitable clause, or any combination of suitable clauses, wherein the nucleic acid sequence consists of at least 97% sequence identity to the sequence of SEQ ID NO: 2.

39. The pharmaceutical composition of clause 23, any other suitable clause, or any combination of suitable clauses, wherein the nucleic acid sequence consists of at least 98% sequence identity to the sequence of SEQ ID NO: 2.

40. The pharmaceutical composition of clause 23, any other suitable clause, or any combination of suitable clauses, wherein the nucleic acid sequence consists of at least 99% sequence identity to the sequence of SEQ ID NO: 2.

41. The pharmaceutical composition of clause 23, any other suitable clause, or any combination of suitable clauses, wherein the nucleic acid sequence provides for an aspartate (D) amino acid at position 228 of the amino acid sequence of SEQ ID NO: 1.

42. The pharmaceutical composition of clause 23, any other suitable clause, or any combination of suitable clauses, wherein the nucleic acid sequence provides for a histidine (H) amino acid at position 365 of the amino acid sequence of SEQ ID NO: 1.

43. The pharmaceutical composition of clause 23, any other suitable clause, or any combination of suitable clauses, wherein the nucleic acid sequence provides for an arginine (R) amino acid at position 388 of the amino acid sequence of SEQ ID NO: 1.

44. The pharmaceutical composition of clause 23, any other suitable clause, or any combination of suitable clauses, wherein the nucleic acid sequence comprises SEQ ID NO: 3.

45. The pharmaceutical composition of clause 23, any other suitable clause, or any combination of suitable clauses, wherein the nucleic acid sequence comprises at least 80% sequence identity to the sequence of SEQ ID NO: 3.

46. The pharmaceutical composition of clause 23, any other suitable clause, or any combination of suitable clauses, wherein the nucleic acid sequence comprises at least 85% sequence identity to the sequence of SEQ ID NO: 3.

47. The pharmaceutical composition of clause 23, any other suitable clause, or any combination of suitable clauses, wherein the nucleic acid sequence comprises at least 90% sequence identity to the sequence of SEQ ID NO: 3.

48. The pharmaceutical composition of clause 23, any other suitable clause, or any combination of suitable clauses, wherein the nucleic acid sequence comprises at least 95% sequence identity to the sequence of SEQ ID NO: 3.

49. The pharmaceutical composition of clause 23, any other suitable clause, or any combination of suitable clauses, wherein the nucleic acid sequence comprises at least 96% sequence identity to the sequence of SEQ ID NO: 3.

50. The pharmaceutical composition of clause 23, any other suitable clause, or any combination of suitable clauses, wherein the nucleic acid sequence comprises at least 97% sequence identity to the sequence of SEQ ID NO: 3.

51. The pharmaceutical composition of clause 23, any other suitable clause, or any combination of suitable clauses, wherein the nucleic acid sequence comprises at least 98% sequence identity to the sequence of SEQ ID NO: 3.

52. The pharmaceutical composition of clause 23, any other suitable clause, or any combination of suitable clauses, wherein the nucleic acid sequence comprises at least 99% sequence identity to the sequence of SEQ ID NO: 3.

53. The pharmaceutical composition of clause 23, any other suitable clause, or any combination of suitable clauses, wherein the nucleic acid sequence consists of at least 80% sequence identity to the sequence of SEQ ID NO: 3.

54. The pharmaceutical composition of clause 23, any other suitable clause, or any combination of suitable clauses, wherein the nucleic acid sequence consists of at least 85% sequence identity to the sequence of SEQ ID NO: 3.

55. The pharmaceutical composition of clause 23, any other suitable clause, or any combination of suitable clauses, wherein the nucleic acid sequence consists of at least 90% sequence identity to the sequence of SEQ ID NO: 3.

56. The pharmaceutical composition of clause 23, any other suitable clause, or any combination of suitable clauses, wherein the nucleic acid sequence consists of at least 95% sequence identity to the sequence of SEQ ID NO: 3.

57. The pharmaceutical composition of clause 23, any other suitable clause, or any combination of suitable clauses, wherein the nucleic acid sequence consists of at least 96% sequence identity to the sequence of SEQ ID NO: 3.

58. The pharmaceutical composition of clause 23, any other suitable clause, or any combination of suitable clauses, wherein the nucleic acid sequence consists of at least 97% sequence identity to the sequence of SEQ ID NO: 3.

59. The pharmaceutical composition of clause 23, any other suitable clause, or any combination of suitable clauses, wherein the nucleic acid sequence consists of at least 98% sequence identity to the sequence of SEQ ID NO: 3.

60. The pharmaceutical composition of clause 23, any other suitable clause, or any combination of suitable clauses, wherein the nucleic acid sequence consists of at least 99% sequence identity to the sequence of SEQ ID NO: 3.

61. The pharmaceutical composition of clause 23, any other suitable clause, or any combination of suitable clauses, wherein the nucleic acid sequence provides for an aspartate (D) amino acid at position 228 of the amino acid sequence of SEQ ID NO: 1.

62. The pharmaceutical composition of clause 23, any other suitable clause, or any combination of suitable clauses, wherein the nucleic acid sequence provides for a histidine (H) amino acid at position 365 of the amino acid sequence of SEQ ID NO: 1.

63. The pharmaceutical composition of clause 23, any other suitable clause, or any combination of suitable clauses, wherein the nucleic acid sequence provides for an arginine (R) amino acid at position 388 of the amino acid sequence of SEQ ID NO: 1.

64. The pharmaceutical composition of clause 23, any other suitable clause, or any combination of suitable clauses, wherein the nucleic acid sequence is a native sequence that has been modified to comprise codons optimized for expression in prokaryotic cells.

65. The pharmaceutical composition of clause 64, any other suitable clause, or any combination of suitable clauses, wherein the nucleic acid sequence comprises SEQ ID NO: 4.

66. The pharmaceutical composition of clause 64, any other suitable clause, or any combination of suitable clauses, wherein the nucleic acid sequence comprises at least 80% sequence identity to the sequence of SEQ ID NO: 4.

67. The pharmaceutical composition of clause 64, any other suitable clause, or any combination of suitable clauses, wherein the nucleic acid sequence comprises at least 85% sequence identity to the sequence of SEQ ID NO: 4.

68. The pharmaceutical composition of clause 64, any other suitable clause, or any combination of suitable clauses, wherein the nucleic acid sequence comprises at least 90% sequence identity to the sequence of SEQ ID NO: 4.

69. The pharmaceutical composition of clause 64, any other suitable clause, or any combination of suitable clauses, wherein the nucleic acid sequence comprises at least 95% sequence identity to the sequence of SEQ ID NO: 4.

70. The pharmaceutical composition of clause 64, any other suitable clause, or any combination of suitable clauses, wherein the nucleic acid sequence comprises at least 96% sequence identity to the sequence of SEQ ID NO: 4.

71. The pharmaceutical composition of clause 64, any other suitable clause, or any combination of suitable clauses, wherein the nucleic acid sequence comprises at least 97% sequence identity to the sequence of SEQ ID NO: 4.

72. The pharmaceutical composition of clause 64, any other suitable clause, or any combination of suitable clauses, wherein the nucleic acid sequence comprises at least 98% sequence identity to the sequence of SEQ ID NO: 4.

73. The pharmaceutical composition of clause 64, any other suitable clause, or any combination of suitable clauses, wherein the nucleic acid sequence comprises at least 99% sequence identity to the sequence of SEQ ID NO: 4.

74. The pharmaceutical composition of clause 64, any other suitable clause, or any combination of suitable clauses, wherein the nucleic acid sequence consists of at least 80% sequence identity to the sequence of SEQ ID NO: 4.

75. The pharmaceutical composition of clause 64, any other suitable clause, or any combination of suitable clauses, wherein the nucleic acid sequence consists of at least 85% sequence identity to the sequence of SEQ ID NO: 4.

76. The pharmaceutical composition of clause 64, any other suitable clause, or any combination of suitable clauses, wherein the nucleic acid sequence consists of at least 90% sequence identity to the sequence of SEQ ID NO: 4.

77. The pharmaceutical composition of clause 64, any other suitable clause, or any combination of suitable clauses, wherein the nucleic acid sequence consists of at least 95% sequence identity to the sequence of SEQ ID NO: 4.

78. The pharmaceutical composition of clause 64, any other suitable clause, or any combination of suitable clauses, wherein the nucleic acid sequence consists of at least 96% sequence identity to the sequence of SEQ ID NO: 4.

79. The pharmaceutical composition of clause 64, any other suitable clause, or any combination of suitable clauses, wherein the nucleic acid sequence consists of at least 97% sequence identity to the sequence of SEQ ID NO: 4.

80. The pharmaceutical composition of clause 64, any other suitable clause, or any combination of suitable clauses, wherein the nucleic acid sequence consists of at least 98% sequence identity to the sequence of SEQ ID NO: 4.

81. The pharmaceutical composition of clause 64, any other suitable clause, or any combination of suitable clauses, wherein the nucleic acid sequence consists of at least 99% sequence identity to the sequence of SEQ ID NO: 4.

82. The pharmaceutical composition of clause 64, any other suitable clause, or any combination of suitable clauses, wherein the nucleic acid sequence provides for an aspartate (D) amino acid at position 228 of the amino acid sequence of SEQ ID NO: 1.

83. The pharmaceutical composition of clause 64, any other suitable clause, or any combination of suitable clauses, wherein the nucleic acid sequence provides for a histidine (H) amino acid at position 365 of the amino acid sequence of SEQ ID NO: 1.

84. The pharmaceutical composition of clause 64, any other suitable clause, or any combination of suitable clauses, wherein the nucleic acid sequence provides for an arginine (R) amino acid at position 388 of the amino acid sequence of SEQ ID NO: 1.

85. The pharmaceutical composition of clause 1, any other suitable clause, or any combination of suitable clauses, wherein the pharmaceutical composition is an oral formulation.

86. The pharmaceutical composition of clause 85, any other suitable clause, or any combination of suitable clauses, wherein the oral formulation is selected from the group consisting of a tablet, a capsule, a suspension, an emulsion, a syrup, a colloidal dispersion, a dispersion, and an effervescent composition.

87. The pharmaceutical composition of clause 85, any other suitable clause, or any combination of suitable clauses, wherein the oral formulation is a suspension.

88. The pharmaceutical composition of clause 85, any other suitable clause, or any combination of suitable clauses, wherein the oral formulation is a reconstitutable suspension.

89. The pharmaceutical composition of clause 1, any other suitable clause, or any combination of suitable clauses, wherein the pharmaceutical composition is a parenteral formulation.

90. The pharmaceutical composition of clause 89, any other suitable clause, or any combination of suitable clauses, wherein the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

91. The pharmaceutical composition of clause 1, any other suitable clause, or any combination of suitable clauses, wherein the pharmaceutical composition further comprises a second therapeutic agent.

92. The pharmaceutical composition of clause 1, any other suitable clause, or any combination of suitable clauses, wherein the pharmaceutical composition is formulated as a single dose.

93. The pharmaceutical composition of clause 1, any other suitable clause, or any combination of suitable clauses, wherein the pharmaceutical composition is formulated as a single unit dose.

94. A method of treating an eye disorder in a patient in need thereof, said method comprising the step of administering a therapeutically effective amount of a pharmaceutical composition to the patient, wherein the pharmaceutical composition comprises i) a MsP1 enzyme and ii) one or more pharmaceutically acceptable carriers.

95. The method of clause 94, any other suitable clause, or any combination of suitable clauses, wherein the patient is an animal.

96. The method of clause 95, any other suitable clause, or any combination of suitable clauses, wherein the animal is a mammal.

97. The method of clause 95, any other suitable clause, or any combination of suitable clauses, wherein the animal is a human.

98. The method of clause 94, any other suitable clause, or any combination of suitable clauses, wherein the eye disorder is age-related macular degeneration (AMD).

99. The method of clause 98, any other suitable clause, or any combination of suitable clauses, wherein the AMD is dry form AMD.

100. The method of clause 98, any other suitable clause, or any combination of suitable clauses, wherein the AMD is wet form AMD.

101. The method of clause 94, any other suitable clause, or any combination of suitable clauses, wherein the administration provides a reduction in one or more symptoms of the eye disorder in the patient.

102. The method of clause 101, any other suitable clause, or any combination of suitable clauses, wherein the one or more symptoms comprise presence of drusen.

103. The method of clause 101, any other suitable clause, or any combination of suitable clauses, wherein the one or more symptoms comprise blurred vision.

104. The method of clause 101, any other suitable clause, or any combination of suitable clauses, wherein the one or more symptoms comprise distorted vision.

105. The method of clause 101, any other suitable clause, or any combination of suitable clauses, wherein the one or more symptoms comprise color perception.

106. The method of clause 94, any other suitable clause, or any combination of suitable clauses, wherein the administration degrades cycloretinal in the patient.

107. The method of clause 106, any other suitable clause, or any combination of suitable clauses, wherein the cycloretinal is degraded to beta-ionone.

108. The method of clause 106, any other suitable clause, or any combination of suitable clauses, wherein the cycloretinal is degraded to 2,4-dimethylbenzaldehyde (2,4-DMB).

109. The method of clause 94, any other suitable clause, or any combination of suitable clauses, wherein the administration provides a reduction in clinical progression of the eye disorder in the patient.

110. The method of clause 94, any other suitable clause, or any combination of suitable clauses, wherein the pharmaceutical composition is administered to the patient at a dose of about 0.001 to about 1000 mg of the MsP1 enzyme per kg of body weight.

111. The method of clause 94, any other suitable clause, or any combination of suitable clauses, wherein the pharmaceutical composition is administered to the patient at a dose of about 0.001 to about 100 mg of the MsP1 enzyme per kg of body weight.

112. The method of clause 94, any other suitable clause, or any combination of suitable clauses, wherein the pharmaceutical composition is administered to the patient at a dose of about 0.001 to about 10 mg of the MsP1 enzyme per kg of body weight.

113. The method of clause 94, any other suitable clause, or any combination of suitable clauses, wherein the pharmaceutical composition is administered to the patient at a dose of about 1 to about 5 mg of the MsP1 enzyme per kg of body weight.

114. The method of clause 94, any other suitable clause, or any combination of suitable clauses, wherein the pharmaceutical composition is administered to the patient at a dose of about 1 mg of the MsP1 enzyme per kg of body weight.

115. The method of clause 94, any other suitable clause, or any combination of suitable clauses, wherein the pharmaceutical composition is administered to the patient at a dose of about 2 mg of the MsP1 enzyme per kg of body weight.

116. The method of clause 94, any other suitable clause, or any combination of suitable clauses, wherein the pharmaceutical composition is administered to the patient at a dose of about 3 mg of the MsP1 enzyme per kg of body weight.

117. The method of clause 94, any other suitable clause, or any combination of suitable clauses, wherein the pharmaceutical composition is administered to the patient at a dose of about 4 mg of the MsP1 enzyme per kg of body weight.

118. The method of clause 94, any other suitable clause, or any combination of suitable clauses, wherein the pharmaceutical composition is administered to the patient at a dose of about 5 mg of the MsP1 enzyme per kg of body weight.

119. The method of clause 94, any other suitable clause, or any combination of suitable clauses, wherein the administration is an oral administration.

120. The method of clause 119, any other suitable clause, or any combination of suitable clauses, wherein the oral administration is selected from the group consisting of a tablet, a capsule, a suspension, an emulsion, a syrup, a colloidal dispersion, a dispersion, and an effervescent composition.

121. The method of clause 119, any other suitable clause, or any combination of suitable clauses, wherein the oral formulation is a suspension.

122. The method of clause 119, any other suitable clause, or any combination of suitable clauses, wherein the oral formulation is a reconstitutable suspension.

123. The method of clause 94, any other suitable clause, or any combination of suitable clauses, wherein the administration is a parenteral administration.

124. The method of clause 123, any other suitable clause, or any combination of suitable clauses, wherein the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

125. The method of clause 94, any other suitable clause, or any combination of suitable clauses, wherein the pharmaceutical composition is administered as a single dose.

126. The method of clause 94, any other suitable clause, or any combination of suitable clauses, wherein the pharmaceutical composition is administered as a single unit dose.

127. The method of clause 94, any other suitable clause, or any combination of suitable clauses, wherein the method further comprises administration of a second therapeutic agent to the patient.

128. The method of clause 127, any other suitable clause, or any combination of suitable clauses, wherein the second therapeutic agent is lutein.

129. The method of clause 127, any other suitable clause, or any combination of suitable clauses, wherein the second therapeutic agent is zeaxanthin.

130. The method of clause 127, any other suitable clause, or any combination of suitable clauses, wherein the second therapeutic agent is zinc.

131. The method of clause 127, any other suitable clause, or any combination of suitable clauses, wherein the second therapeutic agent is Vitamin C.

132. The method of clause 127, any other suitable clause, or any combination of suitable clauses, wherein the second therapeutic agent is Vitamin E.

133. A method of reducing cycloretinal in a patient in need thereof, said method comprising the step of administering a therapeutically effective amount of a pharmaceutical composition to the patient, wherein the pharmaceutical composition comprises i) a MsP1 enzyme or a variant thereof and ii) one or more pharmaceutically acceptable carriers.

134. The method of clause 133, any other suitable clause, or any combination of suitable clauses, wherein the cycloretinal is reduced in an eye of the patient.

135. The method of clause 134, any other suitable clause, or any combination of suitable clauses, wherein the eye comprises a retina, and wherein the cycloretinal is reduced in the retina.

136. The method of clause 135, any other suitable clause, or any combination of suitable clauses, wherein the retina comprises a retinal pigment epithelium, and wherein the cycloretinal is reduced in the retinal pigment epithelium.

137. The method of clause 133, any other suitable clause, or any combination of suitable clauses, wherein the administration degrades cycloretinal in the patient.

138. The method of clause 137, any other suitable clause, or any combination of suitable clauses, wherein the cycloretinal is degraded to beta-ionone.

139. The method of clause 137, any other suitable clause, or any combination of suitable clauses, wherein the cycloretinal is degraded to 2,4-dimethylbenzaldehyde (2,4-DMB).

140. The method of clause 133, any other suitable clause, or any combination of suitable clauses, wherein the administration provides a reduction in clinical progression of the eye disorder in the patient.

141. The method of clause 133, any other suitable clause, or any combination of suitable clauses, wherein the pharmaceutical composition is administered to the patient at a dose of about 0.001 to about 1000 mg of the MsP1 enzyme per kg of body weight.

142. The method of clause 133, any other suitable clause, or any combination of suitable clauses, wherein the pharmaceutical composition is administered to the patient at a dose of about 0.001 to about 100 mg of the MsP1 enzyme per kg of body weight.

143. The method of clause 133, any other suitable clause, or any combination of suitable clauses, wherein the pharmaceutical composition is administered to the patient at a dose of about 0.001 to about 10 mg of the MsP1 enzyme per kg of body weight.

144. The method of clause 133, any other suitable clause, or any combination of suitable clauses, wherein the pharmaceutical composition is administered to the patient at a dose of about 1 to about 5 mg of the MsP1 enzyme per kg of body weight.

145. The method of clause 133, any other suitable clause, or any combination of suitable clauses, wherein the pharmaceutical composition is administered to the patient at a dose of about 1 mg of the MsP1 enzyme per kg of body weight.

146. The method of clause 133, any other suitable clause, or any combination of suitable clauses, wherein the pharmaceutical composition is administered to the patient at a dose of about 2 mg of the MsP1 enzyme per kg of body weight.

147. The method of clause 133, any other suitable clause, or any combination of suitable clauses, wherein the pharmaceutical composition is administered to the patient at a dose of about 3 mg of the MsP1 enzyme per kg of body weight.

148. The method of clause 133, any other suitable clause, or any combination of suitable clauses, wherein the pharmaceutical composition is administered to the patient at a dose of about 4 mg of the MsP1 enzyme per kg of body weight.

149. The method of clause 133, any other suitable clause, or any combination of suitable clauses, wherein the pharmaceutical composition is administered to the patient at a dose of about 5 mg of the MsP1 enzyme per kg of body weight.

150. The method of clause 133, any other suitable clause, or any combination of suitable clauses, wherein the administration is an oral administration.

151. The method of clause 150, any other suitable clause, or any combination of suitable clauses, wherein the oral administration is selected from the group consisting of a tablet, a capsule, a suspension, an emulsion, a syrup, a colloidal dispersion, a dispersion, and an effervescent composition.

152. The method of clause 150, any other suitable clause, or any combination of suitable clauses, wherein the oral formulation is a suspension.

153. The method of clause 150, any other suitable clause, or any combination of suitable clauses, wherein the oral formulation is a reconstitutable suspension.

154. The method of clause 133, any other suitable clause, or any combination of suitable clauses, wherein the administration is a parenteral administration.

155. The method of clause 154, any other suitable clause, or any combination of suitable clauses, wherein the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

156. The method of clause 133, any other suitable clause, or any combination of suitable clauses, wherein the pharmaceutical composition is administered as a single dose.

157. The method of clause 133, any other suitable clause, or any combination of suitable clauses, wherein the pharmaceutical composition is administered as a single unit dose.

158. The method of clause 133, any other suitable clause, or any combination of suitable clauses, wherein the method further comprises administration of a second therapeutic agent to the patient.

159. The method of clause 158, any other suitable clause, or any combination of suitable clauses, wherein the second therapeutic agent is lutein.

160. The method of clause 158, any other suitable clause, or any combination of suitable clauses, wherein the second therapeutic agent is zeaxanthin.

161. The method of clause 158, any other suitable clause, or any combination of suitable clauses, wherein the second therapeutic agent is zinc.

162. The method of clause 158, any other suitable clause, or any combination of suitable clauses, wherein the second therapeutic agent is Vitamin C.

163. The method of clause 158, any other suitable clause, or any combination of suitable clauses, wherein the second therapeutic agent is Vitamin E.

164. A recombinant polynucleotide comprising a heterologous regulatory element operably linked to a nucleic acid sequence encoding a MsP1 protein.

165. The recombinant polynucleotide of clause 164, any other suitable clause, or any combination of suitable clauses, wherein the MsP1 protein comprises SEQ ID NO: 1.

166. The recombinant polynucleotide of clause 164, any other suitable clause, or any combination of suitable clauses, wherein the MsP1 protein comprises at least 80% sequence identity to the sequence of SEQ ID NO: 1.

167. The recombinant polynucleotide of clause 164, any other suitable clause, or any combination of suitable clauses, wherein the MsP1 protein comprises at least 85% sequence identity to the sequence of SEQ ID NO: 1.

168. The recombinant polynucleotide of clause 164, any other suitable clause, or any combination of suitable clauses, wherein the MsP1 protein comprises at least 90% sequence identity to the sequence of SEQ ID NO: 1.

169. The recombinant polynucleotide of clause 164, any other suitable clause, or any combination of suitable clauses, wherein the MsP1 protein comprises at least 95% sequence identity to the sequence of SEQ ID NO: 1.

170. The recombinant polynucleotide of clause 164, any other suitable clause, or any combination of suitable clauses, wherein the MsP1 protein comprises at least 96% sequence identity to the sequence of SEQ ID NO: 1.

171. The recombinant polynucleotide of clause 164, any other suitable clause, or any combination of suitable clauses, wherein the MsP1 protein comprises at least 97% sequence identity to the sequence of SEQ ID NO: 1.

172. The recombinant polynucleotide of clause 164, any other suitable clause, or any combination of suitable clauses, wherein the MsP1 protein comprises at least 98% sequence identity to the sequence of SEQ ID NO: 1.

173. The recombinant polynucleotide of clause 164, any other suitable clause, or any combination of suitable clauses, wherein the MsP1 protein comprises at least 99% sequence identity to the sequence of SEQ ID NO: 1.

174. The recombinant polynucleotide of clause 164, any other suitable clause, or any combination of suitable clauses, wherein the MsP1 protein consists of at least 80% sequence identity to the sequence of SEQ ID NO: 1.

175. The recombinant polynucleotide of clause 164, any other suitable clause, or any combination of suitable clauses, wherein the MsP1 protein consists of at least 85% sequence identity to the sequence of SEQ ID NO: 1.

176. The recombinant polynucleotide of clause 164, any other suitable clause, or any combination of suitable clauses, wherein the MsP1 protein consists of at least 90% sequence identity to the sequence of SEQ ID NO: 1.

177. The recombinant polynucleotide of clause 164, any other suitable clause, or any combination of suitable clauses, wherein the MsP1 protein consists of at least 95% sequence identity to the sequence of SEQ ID NO: 1.

178. The recombinant polynucleotide of clause 164, any other suitable clause, or any combination of suitable clauses, wherein the MsP1 protein consists of at least 96% sequence identity to the sequence of SEQ ID NO: 1.

179. The recombinant polynucleotide of clause 164, any other suitable clause, or any combination of suitable clauses, wherein the MsP1 protein consists of at least 97% sequence identity to the sequence of SEQ ID NO: 1.

180. The recombinant polynucleotide of clause 164, any other suitable clause, or any combination of suitable clauses, wherein the MsP1 protein consists of at least 98% sequence identity to the sequence of SEQ ID NO: 1.

181. The recombinant polynucleotide of clause 164, any other suitable clause, or any combination of suitable clauses, wherein the MsP1 protein consists of at least 99% sequence identity to the sequence of SEQ ID NO: 1.

182. The recombinant polynucleotide of clause 164, any other suitable clause, or any combination of suitable clauses, wherein the MsP1 protein comprises an aspartate (D) amino acid at position 228 of SEQ ID NO: 1.

183. The recombinant polynucleotide of clause 164, any other suitable clause, or any combination of suitable clauses, wherein the MsP1 protein comprises a histidine (H) amino acid at position 365 of SEQ ID NO: 1.

184. The recombinant polynucleotide of clause 164, any other suitable clause, or any combination of suitable clauses, wherein the the MsP1 protein comprises an arginine (R) amino acid at position 388 of SEQ ID NO: 1.

185. The recombinant polynucleotide of clause 164, any other suitable clause, or any combination of suitable clauses, wherein the heterologous regulatory element is a promoter that functions in prokaryotic cells.

186. The recombinant polynucleotide of clause 164, any other suitable clause, or any combination of suitable clauses, wherein the nucleic acid sequence comprises SEQ ID NO: 2.

187. The recombinant polynucleotide of clause 164, any other suitable clause, or any combination of suitable clauses, wherein the nucleic acid sequence comprises at least 80% sequence identity to the sequence of SEQ ID NO: 2.

188. The recombinant polynucleotide of clause 164, any other suitable clause, or any combination of suitable clauses, wherein the nucleic acid sequence comprises at least 85% sequence identity to the sequence of SEQ ID NO: 2.

189. The recombinant polynucleotide of clause 164, any other suitable clause, or any combination of suitable clauses, wherein the nucleic acid sequence comprises at least 90% sequence identity to the sequence of SEQ ID NO: 2.

190. The recombinant polynucleotide of clause 164, any other suitable clause, or any combination of suitable clauses, wherein the nucleic acid sequence comprises at least 95% sequence identity to the sequence of SEQ ID NO: 2.

191. The recombinant polynucleotide of clause 164, any other suitable clause, or any combination of suitable clauses, wherein the nucleic acid sequence comprises at least 96% sequence identity to the sequence of SEQ ID NO: 2.

192. The recombinant polynucleotide of clause 164, any other suitable clause, or any combination of suitable clauses, wherein the nucleic acid sequence comprises at least 97% sequence identity to the sequence of SEQ ID NO: 2.

193. The recombinant polynucleotide of clause 164, any other suitable clause, or any combination of suitable clauses, wherein the nucleic acid sequence comprises at least 98% sequence identity to the sequence of SEQ ID NO: 2.

194. The recombinant polynucleotide of clause 164, any other suitable clause, or any combination of suitable clauses, wherein the nucleic acid sequence comprises at least 99% sequence identity to the sequence of SEQ ID NO: 2.

195. The recombinant polynucleotide of clause 164, any other suitable clause, or any combination of suitable clauses, wherein the nucleic acid sequence consists of at least 80% sequence identity to the sequence of SEQ ID NO: 2.

196. The recombinant polynucleotide of clause 164, any other suitable clause, or any combination of suitable clauses, wherein the nucleic acid sequence consists of at least 85% sequence identity to the sequence of SEQ ID NO: 2.

197. The recombinant polynucleotide of clause 164, any other suitable clause, or any combination of suitable clauses, wherein the nucleic acid sequence consists of at least 90% sequence identity to the sequence of SEQ ID NO: 2.

198. The recombinant polynucleotide of clause 164, any other suitable clause, or any combination of suitable clauses, wherein the nucleic acid sequence consists of at least 95% sequence identity to the sequence of SEQ ID NO: 2.

199. The recombinant polynucleotide of clause 164, any other suitable clause, or any combination of suitable clauses, wherein the nucleic acid sequence consists of at least 96% sequence identity to the sequence of SEQ ID NO: 2.

200. The recombinant polynucleotide of clause 164, any other suitable clause, or any combination of suitable clauses, wherein the nucleic acid sequence consists of at least 97% sequence identity to the sequence of SEQ ID NO: 2.

201. The recombinant polynucleotide of clause 164, any other suitable clause, or any combination of suitable clauses, wherein the nucleic acid sequence consists of at least 98% sequence identity to the sequence of SEQ ID NO: 2.

202. The recombinant polynucleotide of clause 164, any other suitable clause, or any combination of suitable clauses, wherein the nucleic acid sequence consists of at least 99% sequence identity to the sequence of SEQ ID NO: 2.

203. The recombinant polynucleotide of clause 164, any other suitable clause, or any combination of suitable clauses, wherein the nucleic acid sequence provides for an aspartate (D) amino acid at position 228 of the amino acid sequence of SEQ ID NO: 1.

204. The recombinant polynucleotide of clause 164, any other suitable clause, or any combination of suitable clauses, wherein the nucleic acid sequence provides for a histidine (H) amino acid at position 365 of the amino acid sequence of SEQ ID NO: 1.

205. The recombinant polynucleotide of clause 164, any other suitable clause, or any combination of suitable clauses, wherein the nucleic acid sequence provides for an arginine (R) amino acid at position 388 of the amino acid sequence of SEQ ID NO: 1.

206. The recombinant polynucleotide of clause 164, any other suitable clause, or any combination of suitable clauses, wherein the nucleic acid sequence comprises SEQ ID NO: 3.

207. The recombinant polynucleotide of clause 164, any other suitable clause, or any combination of suitable clauses, wherein the nucleic acid sequence comprises at least 80% sequence identity to the sequence of SEQ ID NO: 3.

208. The recombinant polynucleotide of clause 164, any other suitable clause, or any combination of suitable clauses, wherein the nucleic acid sequence comprises at least 85% sequence identity to the sequence of SEQ ID NO: 3.

209. The recombinant polynucleotide of clause 164, any other suitable clause, or any combination of suitable clauses, wherein the nucleic acid sequence comprises at least 90% sequence identity to the sequence of SEQ ID NO: 3.

210. The recombinant polynucleotide of clause 164, any other suitable clause, or any combination of suitable clauses, wherein the nucleic acid sequence comprises at least 95% sequence identity to the sequence of SEQ ID NO: 3.

211. The recombinant polynucleotide of clause 164, any other suitable clause, or any combination of suitable clauses, wherein the nucleic acid sequence comprises at least 96% sequence identity to the sequence of SEQ ID NO: 3.

212. The recombinant polynucleotide of clause 164, any other suitable clause, or any combination of suitable clauses, wherein the nucleic acid sequence comprises at least 97% sequence identity to the sequence of SEQ ID NO: 3.

213. The recombinant polynucleotide of clause 164, any other suitable clause, or any combination of suitable clauses, wherein the nucleic acid sequence comprises at least 98% sequence identity to the sequence of SEQ ID NO: 3.

214. The recombinant polynucleotide of clause 164, any other suitable clause, or any combination of suitable clauses, wherein the nucleic acid sequence comprises at least 99% sequence identity to the sequence of SEQ ID NO: 3.

215. The recombinant polynucleotide of clause 164, any other suitable clause, or any combination of suitable clauses, wherein the nucleic acid sequence consists of at least 80% sequence identity to the sequence of SEQ ID NO: 3.

216. The recombinant polynucleotide of clause 164, any other suitable clause, or any combination of suitable clauses, wherein the nucleic acid sequence consists of at least 85% sequence identity to the sequence of SEQ ID NO: 3.

217. The recombinant polynucleotide of clause 164, any other suitable clause, or any combination of suitable clauses, wherein the nucleic acid sequence consists of at least 90% sequence identity to the sequence of SEQ ID NO: 3.

218. The recombinant polynucleotide of clause 164, any other suitable clause, or any combination of suitable clauses, wherein the nucleic acid sequence consists of at least 95% sequence identity to the sequence of SEQ ID NO: 3.

219. The recombinant polynucleotide of clause 164, any other suitable clause, or any combination of suitable clauses, wherein the nucleic acid sequence consists of at least 96% sequence identity to the sequence of SEQ ID NO: 3.

220. The recombinant polynucleotide of clause 164, any other suitable clause, or any combination of suitable clauses, wherein the nucleic acid sequence consists of at least 97% sequence identity to the sequence of SEQ ID NO: 3.

221. The recombinant polynucleotide of clause 164, any other suitable clause, or any combination of suitable clauses, wherein the nucleic acid sequence consists of at least 98% sequence identity to the sequence of SEQ ID NO: 3.

222. The recombinant polynucleotide of clause 164, any other suitable clause, or any combination of suitable clauses, wherein the nucleic acid sequence consists of at least 99% sequence identity to the sequence of SEQ ID NO: 3.

223. The recombinant polynucleotide of clause 222, any other suitable clause, or any combination of suitable clauses, wherein the nucleic acid sequence provides for an aspartate (D) amino acid at position 228 of the amino acid sequence of SEQ ID NO: 1.

224. The recombinant polynucleotide of clause 222, any other suitable clause, or any combination of suitable clauses, wherein the nucleic acid sequence provides for a histidine (H) amino acid at position 365 of the amino acid sequence of SEQ ID NO: 1.

225. The recombinant polynucleotide of clause 222, any other suitable clause, or any combination of suitable clauses, wherein the nucleic acid sequence provides for an arginine (R) amino acid at position 388 of the amino acid sequence of SEQ ID NO: 1.

226. The recombinant polynucleotide of clause 164, any other suitable clause, or any combination of suitable clauses, wherein the nucleic acid sequence is a native sequence that has been modified to comprise codons optimized for expression in prokaryotic cells.

227. The recombinant polynucleotide of clause 226, any other suitable clause, or any combination of suitable clauses, wherein the nucleic acid sequence comprises SEQ ID NO: 4.

228. The recombinant polynucleotide of clause 226, any other suitable clause, or any combination of suitable clauses, wherein the nucleic acid sequence comprises at least 80% sequence identity to the sequence of SEQ ID NO: 4.

229. The recombinant polynucleotide of clause 226, any other suitable clause, or any combination of suitable clauses, wherein the nucleic acid sequence comprises at least 85% sequence identity to the sequence of SEQ ID NO: 4.

230. The recombinant polynucleotide of clause 226, any other suitable clause, or any combination of suitable clauses, wherein the nucleic acid sequence comprises at least 90% sequence identity to the sequence of SEQ ID NO: 4.

231. The recombinant polynucleotide of clause 226, any other suitable clause, or any combination of suitable clauses, wherein the nucleic acid sequence comprises at least 95% sequence identity to the sequence of SEQ ID NO: 4.

232. The recombinant polynucleotide of clause 226, any other suitable clause, or any combination of suitable clauses, wherein the nucleic acid sequence comprises at least 96% sequence identity to the sequence of SEQ ID NO: 4.

233. The recombinant polynucleotide of clause 226, any other suitable clause, or any combination of suitable clauses, wherein the nucleic acid sequence comprises at least 97% sequence identity to the sequence of SEQ ID NO: 4.

234. The recombinant polynucleotide of clause 226, any other suitable clause, or any combination of suitable clauses, wherein the nucleic acid sequence comprises at least 98% sequence identity to the sequence of SEQ ID NO: 4.

235. The recombinant polynucleotide of clause 226, any other suitable clause, or any combination of suitable clauses, wherein the nucleic acid sequence comprises at least 99% sequence identity to the sequence of SEQ ID NO: 4.

236. The recombinant polynucleotide of clause 226, any other suitable clause, or any combination of suitable clauses, wherein the nucleic acid sequence consists of at least 80% sequence identity to the sequence of SEQ ID NO: 4.

237. The recombinant polynucleotide of clause 226, any other suitable clause, or any combination of suitable clauses, wherein the nucleic acid sequence consists of at least 85% sequence identity to the sequence of SEQ ID NO: 4.

238. The recombinant polynucleotide of clause 226, any other suitable clause, or any combination of suitable clauses, wherein the nucleic acid sequence consists of at least 90% sequence identity to the sequence of SEQ ID NO: 4.

239. The recombinant polynucleotide of clause 226, any other suitable clause, or any combination of suitable clauses, wherein the nucleic acid sequence consists of at least 95% sequence identity to the sequence of SEQ ID NO: 4.

240. The recombinant polynucleotide of clause 226, any other suitable clause, or any combination of suitable clauses, wherein the nucleic acid sequence consists of at least 96% sequence identity to the sequence of SEQ ID NO: 4.

241. The recombinant polynucleotide of clause 226, any other suitable clause, or any combination of suitable clauses, wherein the nucleic acid sequence consists of at least 97% sequence identity to the sequence of SEQ ID NO: 4.

242. The recombinant polynucleotide of clause 226, any other suitable clause, or any combination of suitable clauses, wherein the nucleic acid sequence consists of at least 98% sequence identity to the sequence of SEQ ID NO: 4.

243. The recombinant polynucleotide of clause 226, any other suitable clause, or any combination of suitable clauses, wherein the nucleic acid sequence consists of at least 99% sequence identity to the sequence of SEQ ID NO: 4.

244. The recombinant polynucleotide of clause 226, any other suitable clause, or any combination of suitable clauses, wherein the nucleic acid sequence provides for an aspartate (D) amino acid at position 228 of the amino acid sequence of SEQ ID NO: 1.

245. The recombinant polynucleotide of clause 226, any other suitable clause, or any combination of suitable clauses, wherein the nucleic acid sequence provides for a histidine (H) amino acid at position 365 of the amino acid sequence of SEQ ID NO: 1.

246. The recombinant polynucleotide of clause 226, any other suitable clause, or any combination of suitable clauses, wherein the nucleic acid sequence provides for an arginine (R) amino acid at position 388 of the amino acid sequence of SEQ ID NO: 1.

247. An expression vector comprising a recombinant polynucleotide of any one of the above clauses and a selectable marker gene.

248. The expression vector of clause 247, any other suitable clause, or any combination of suitable clauses, wherein the selectable marker gene encodes for antibiotic resistance.

249. The expression vector of clause 248, any other suitable clause, or any combination of suitable clauses, wherein the selectable marker gene is neomycin (Neo).

250. The expression vector of clause 248, any other suitable clause, or any combination of suitable clauses, wherein the selectable marker gene is bleomycin (Ble).

251. The expression vector of clause 248, any other suitable clause, or any combination of suitable clauses, wherein the selectable marker gene is blasticidin (Bsr/Bsd).

252. The expression vector of clause 248, any other suitable clause, or any combination of suitable clauses, wherein the selectable marker gene confers neomycin resistance.

253. The expression vector of clause 248, any other suitable clause, or any combination of suitable clauses, wherein the selectable marker gene confers bleomycin resistance.

254. The expression vector of clause 248, any other suitable clause, or any combination of suitable clauses, wherein the selectable marker gene confers blasticidin resistance.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Materials and Methods

For the examples described herein, the following materials were utilized. All-trans retinal and L-proline were obtained from Sigma-Aldrich. The silica (SiliaFlash® F60; particle size: 40-63 μm, 230-400 mesh) was obtained from Silicycle Inc, Canada. The resin, Amberlite® weakly acidic cation exchanger, hydrogen form, CG50 was also obtained from Sigma-Aldrich.

Chemical compounds and media were acquired from the DIFCO laboratories (Detroit, Michigan, USA), Fluka (Germany), MP Biomedicals, LLC (France) Sigma-Aldrich and Merck (Darmstadt, Germany). NMR spectra were acquired by Bruker AvanceCore 400 MHz instrument. GCMS data was acquired by Thermo Electron GC Ultra with DSQ instrument. LCMS data was acquired by Thermo Scientific Q Exactive Focus coupled with UltiMate 3000 Rapid Separation LC instrument.

Fungal strain Marasmius scorodonius 3975 (ATCC Number 76439) was obtained from American Type Culture Collection Center (ATCC). Further, all culture media were obtained from Difco™ and prepared according to manufacturer instructions. YM agar and YM broth (ATCC® medium 200) was used for the growth of Marasmius scorodonius 3975.

### Example 2

### Synthesis of Cycloretinal

Cycloretinal was prepared. A mixture of 100 mg all-trans retinal (0.3 mmol), 62 mg L-proline (0.5 mol), and 400 μL triethylamine (2.9 mmol) were suspended in ethanol and stirred at room temperature for 24 hours. The crude mixture was purified by flash column chromatography (1:19 EtoAc:Hex). The purified product was characterized by ¹H NMR.

### Example 3

Cycloretinal Biodegradation by Marasmius scorodonius

M-9 medium (5 mL each) was inoculated with Marasmius scorodonius. Cycloretinal (1 μg/mL) was used as the only carbon and energy source and the culture was then incubated at 30° C. and 250 rpm for 24 hours. M-9 medium (5 mL each) with cycloretinal and without Marasmius scorodonius inoculation served as the negative control.

After 24 hours of incubation, broth cultures were transferred to sterile Eppendorf tubes and the organics extracted using an equal volume of dichloromethane (DCM) through vigorous vortexing. The organics were separated by centrifugation (13,000 rpm for 20 minutes), retrieved with a needle, and subsequently concentrated under nitrogen. The dried product was solubilized in 20 μL DCM and analyzed by high performance liquid chromatography (HPLC). HPLC analysis was performed on a Varian Prostar system equipped with a Phenomenex 5 μm 100 Å normal phase column (250×10 mm×5 micron), a UV detector set at 320 nm wavelength and mobile phase comprising 97% DCM and 3% methanol with flow rate of 2 mL/min. Products eluted were further analyzed by GC-MS.

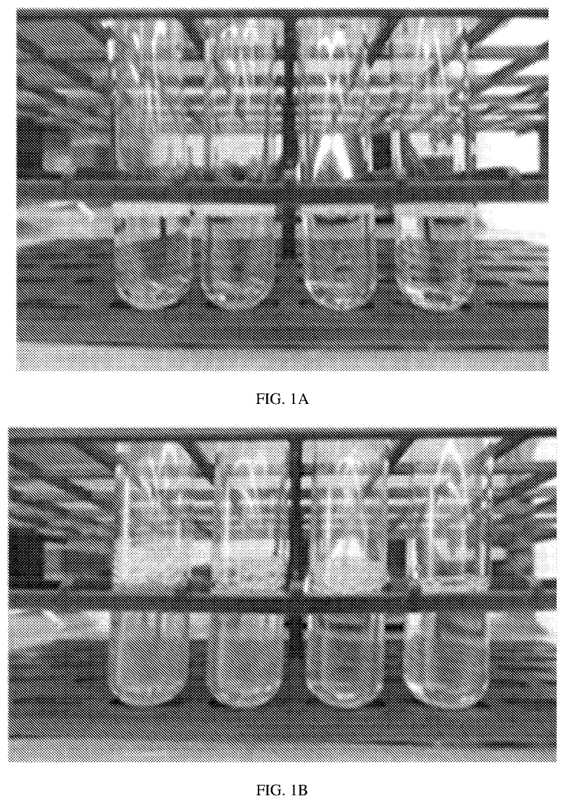

FIGS. 1A-1B compare incubation of purified MsP1 with cycloretinal. Clearance of cycloretinal was observed by visual inspection within 2 hours of incubation (FIG. 1B).

### Example 4

### Cloning, Overexpression and Purification of MsP1 and Variants of MsP1 Cloning

The gene encoding Marasmius scorodonius extracellular peroxidase MsP1 (UniProtKB, B0BK71) and its R388L variant were chemically synthesized with codon optimization for E. coli, having an optimized sequence length 1539 bp cloned into pUC57 vector with a GC content of 54.67% (SEQ ID NO: 5). Synthesized genes were cloned using primers listed in Table 1 below.

TABLE 1
Primers

| | | SEQ |
|---|---|---|
| Primer | | ID |
| Name | Sequence | NO: |
| BamHI- | GGATCCCACCACCACCACCACCAC | SEQ |
| MsP1-F | AAACTGTTCTCCGCAT | ID |
| | | NO: |
| | | 5 |
| HindIII- | AAGCTTTTAAACGGACAGGACGGT | SEQ |
| MsP1-R | ACGGATAGCC | ID |
| | | NO: |
| | | 6 |
| H221A | TCCGGGTGATCAGCAAGGTGCAGA | SEQ |
| MsP1-F | ACACTTTGGCTTCATGG | ID |
| | | NO: |
| | | 7 |
| H221A | CCATGAAGCCAAAGTGTTCTGCAC | SEQ |
| MsP1-R | CTTGCTGATCACCCGGA | ID |
| | | NO: |
| | | 8 |
| D336A | CCCGCTGGTTGATGCACCGGTCCT | SEQ |
| MsP1-F | GGCAGC | ID |
| | | NO: |
| | | 9 |
| D336A | GCTGCCAGGACCGGTGCATCAACC | SEQ |
| MsP1-R | AGCGGG | ID |
| | | NO: |
| | | 10 |

PCR amplification was carried out using Phusion DNA polymerase (New England Biolabs). Primer annealing/extension was carried out at 72° C. for 60 sec. A total of 35 cycles were used. PCR amplified product was run on 1% agarose gel, at 100 Volts for 20 minutes. A 1.5 kb MsP1 band was excised out of gel and gel extraction was performed by using QIA QIAquick® Gel Extraction Kit (250) (Qiagen Sciences Maryland, USA). Gel purified MsP1 was digested with BamHI and HindIII and ligated into the vector pMALc4x (Novagen/New England Biolabs) downstream of the gene coding for maltose binding protein. The genes encoding for Maltose Binding Protein (MBP) and MsP1 are separated by a cleavage site for TEV protease. Cloned constructs were confirmed by sequencing analysis.

### Site Directed Mutagenesis

MsP1 mutants were generated using the Agilent mutagenesis kit (Quick change II site directed mutagenesis kit, catalog number: 200523) and primers of Table 1. Primer annealing was carried out at 55° C. for 60 seconds followed by a polymerization step carried out at 68° C. for 5 minutes. A total of 16 cycles were carried out. After PCR amplification DpnI digestion was performed by adding 1.5 μL DpnI to PCR amplification mixture incubating for 4 hours at 37° C. The resulting mixture was cleaned using resin provided in the kit and transformed into XL1 Blue electrocompetent cells. The resulting clones were screened and sequenced to confirm mutagenesis.

### Overexpression and Purification of MBP-MsP1

Fusion protein (MBP-MsP1) was expressed in BL21 (λDE3, Novagen) transformed with pMALc4x-MBP-MsP1 construct, grown in Luria-Bertani (LB) medium supplemented with ampicillin (100 μg/mL) at 37° C. The culture was grown to an OD600 of 0.7 and at which point 100 μM IPTG and 15 μM hemin (Sigma-Aldrich) were added to the to the culture medium. The culture was subsequently incubated at 16° C. for an additional 24 hours before being harvested by centrifugation (6500×g for 15 minutes). Harvested cells were suspended in column buffer (50 mM tris, 200 mM NaCl, 1 mM EDTA, 10% Glycerol, 1 mM PMSF, 1 mM DDT) at a ratio of 5 mL of buffer per gram of cells and lysed using an ultrasonic homogenizer (Q500 Sonicator, QSonica, LLC). At 50% power, 15×1 second pulses were used to lyse 1 gram of cells. Cell debris was removed by centrifugation (12500×g for 90 min, 4° C.), and the supernatant recovered. The recombinant fusion protein (MBP-MsP1) was purified at 4° C. by passing it through an amylose resin (NEB) column. The crude protein mixture was loaded slowly and then washed with 3 column volumes of column buffer. The clean protein was eluted with 3 column volumes of elution buffer (1M Methyl-D-Glucopyranoside in column buffer). Protein concentration was determined by Bradford assay, using bovine serum albumin as a standard.

### Example 5

### Overexpression and Purification of MBP-TEV Protease

### Overexpression and Purification of MBP-TEV Protease

The construct expressing an autolysis-resistant S219V variant of MBP tagged TEV protease (addgene Plasmid #8835, GenBank, APA32020) was transformed into BL21 DE3 cells along with the tRNA accessory plasmid pRIL (from the BL21 CodonPlus strain, Stratagene) by electroporation. The cells were plated on LB-agar medium containing chloramphenicol (30 μg/mL) and ampicillin (100 μg/mL) and incubated overnight. An individual colony was then selected and grown overnight in 15 mL of LB medium containing chloramphenicol (30 μg/mL) and ampicillin (100 μg/mL) at 37° C. This starter culture was used to inoculate 1 L of LB Miller broth containing 0.2% glucose, chloramphenicol (30 μg/mL) and ampicillin (100 μg/mL). The glucose helps suppress endogenous amylases, thus helping increase protein expression. The culture was shaken (225 rpm) at 37° C. to an optical density of 0.8 at OD 600 nm. This culture was cooled to 30° C. and induced with 1 mM IPTG for 4 hours. The cells were pelleted (7,000 rpm, 10 minutes), resuspended in 25 mL column buffer (50 mM Tris-HCl pH 7.5, 200 mM NaCl, 1 mM EDTA, 10% glycerol), and stored frozen at −80° C. The MBP-tagged TEV protease was purified using the protocol that was used to purify the BLG fusion protein. The presence of the protein was confirmed by SDS-PAGE analysis. The concentration was assessed by Bradford assay using commercial Bovine Serum Albumin (BSA) as a standard.

### TEV Cleavage Reaction and Further Purification

Elution buffer (Methyl-D-glucopyranoside 10%) was diafiltered into 50 mM Tris-HCl buffer containing 200 mM NaCl by using centrifugal units (Pall Corporation) prior to setting up the cleavage reaction. A cleavage reaction was carried out according to previously published procedure in 50 mL total volume containing equal concentration of both proteins (5 mg of each protein, i.e. MBP-MsP1 and MBP-TEV protease) in 50 mM tris-HCL buffer containing 200 mM NaCl, and 1 mL of 1 M Dithiothreitol (DTT) at 4° C. for 5 days. The cleaved, recombinant MsP1 protein with an N-terminal hexa-Histidine tag was purified at 4° C. by passing through a HisTrap FF crude column (GE Healthcare). The bound protein was washed with 20 mM imidazole (500 mL), 100 mM imidazole (50 mL) and then eluted with 250 mM imidazole (30 mL). Histrap purified protein was then passed through amylose resin column to remove any remaining fusion protein. The protein was characterized by SDS-PAGE analysis and in-gel proteolytic (trypsin) digestion along with peptide mass mapping.

### Example 6

### MsP1 Catalyzed Degradation of Cycloretinal

### MsP1 Catalyzed Biotransformation Reactions

Substrate emulsions (0.01%) were prepared, and Tween 40 was used to enhance solubilization of compounds, or otherwise dissolved in ethanol or DMSO. The MsP1 catalyzed transformation was performed with 1 mg of substrate in a total volume of 3 mL of phosphate buffered saline (PBS) (0.01M phosphate buffer with 0.0027M KCl, 0.137M NaCl, and pH 7.4) at 37° C., 250 rpm for 60 min. 0.1 mg of recombinant MsP1 and 2 μL of 20 mM H₂O₂ were added to carry out transformation. Controls were run with all samples, without the addition of enzyme. Samples were withdrawn at five different time points (i.e. 1 min, 5 min, 20 min, 2 h and 12 h). Extraction, analysis, and characterization were performed as detailed in Example 3.

### Standard Curve for Degradation Products

To generate a standard curve for the degradation products, different concentrations of 2,4-dimethyl benzaldehyde (2, 4, 6, 8, 10 μg/20 μL) and β-ionone (1, 2, 3, 4 μg/20 μL) were analyzed by HPLC under the same conditions as as detailed in Example 3.

### Trapping α-Oxoaldehyde Intermediates of the MsP1 Catalyzed Biotransformation Reaction

Substrate was dissolved in DMSO (5% v/v). The MsP1 catalyzed transformation was performed with 2 mg of substrate in a total volume of 8 mL of phosphate buffer saline (PBS) (0.01M phosphate buffer with 0.0027M KCl, 0.137M NaCl, and pH 7.4) at 30° C., 250 rpm for 16 hours. 1 μM of recombinant MsP1 and 16 μL of 20 mM H₂O₂ were added to carry out transformation. Following incubation, the enzyme was removed from the aqueous phase by centrifugation with a 10 kDa PES centrifugal filter. The aqueous phase was incubated with aminoguanidine (10 mg) in a hot water bath (37° C. for 2 hours). Water insoluble products generated from the enzymatic assay was solubilized with CHCl₃ and combined with the organic fractions. The organic fractions were generated and collected from the aminoguanidine coupling reaction/separatory funnel workup (3×10 mL CHCl₃, 1×10 mL brine, keep organic fractions). Organic fractions were concentrated in vacuo and resuspended in methanol (2 mL, HPLC grade) before submission for LCMS and LC-MSMS.

### Activity of MsP1 at Different pH

To determine the optimum pH for MsP1 activity, cycloretinal degradation reactions were carried out in PBS buffer as described herein but at different pH values by adjusting pH with either NaOH or HCl (i.e. a pH of 5.5, 6.4, 7.0, 7.5). Sample OD values were monitored using a UV-Vis spectrophotometer (Genesys™2, ThermoSpectronic) at 430 nm over a period of 300 seconds.

### Enzyme Kinetics

For enzyme kinetics measurements, MsP1 (or MsP1 variants) catalyzed biotransformation of cycloretinal was assessed by monitoring a decrease in absorbance at 430 nm (due to the disappearance of cycloretinal over time). Measurements were conducted in triplicate with a UV-Vis spectrophotometer (Genesys™, ThermoSpectronic) at 430 nm over a period of 500 seconds.

The reaction mixture contained 823 μL PBS (0.01M phosphate buffer with 0.0027M KCl, 0.137M NaCl, and pH 7.4), 50 μL cycloretinal solutions prepared in DMSO, 125 μL MsP1-solution (wild-type or MsP1 variant, 0.05 mg/mL in PBS i.e 0.909 μM) and 2 μL 20 mM H₂O₂. Different substrate concentrations used were, 0.2 mg/mL (90.83972 μM), 0.175 mg/mL (136.2596 μM), 0.15 mg/mL (181.6794 μM), 0.125 mg/mL (227.0993 μM), 0.1 mg/mL (272.5192 μM), 0.075 mg/mL (317.939 μM) and 0.05 mg/mL (363.3589 μM). After substrate delivery, total DMSO concentration was 5% v/v.

One unit of enzyme activity was defined as the amount of enzyme oxidizing 1 μmol of substrate per minute. The initial velocities plotted according to Lineweaver and Burk (Lineweaver, 1934) gave the Michaelis-Menten parameters Vₘₐₓ, K_{M} and k_{cat}.

### Example 7

### Synthesis of Liposomal Cycloretinal, β-Ionone, 2,4-Dimethylbenzaldehyde, Glyoxal, Methylglyoxal, and β-Cyclocitral

The liposomal complexes were prepared as follows. Cycloretinal (10 mg), β-Ionone (20 mg), and 2,4-Dimethylbenzaldehyde (20 mg) were dissolved separately in ethanol (1 mL). Glyoxal (5.80 mg), methylglyoxal (7.21 mg), and β-cyclocitral (15.2 mg) were dissolved separately in chloroform (1 mL). A liposome solution of cholesterol (112 mg), Tween 80 (504 mg), and L-α-phosphatidylcholine (730 mg) were dissolved in 16 mL of chloroform. Liposome solution (2 mL) was added to each of the compounds. The solvents were removed under vacuum and charged with nitrogen. Each of the flasks were then mixed with 5 mL of water and stirred at 60° C. for 1 h. Each solution was placed on ice and sonicated with a probe sonicator (15 seconds on/15 seconds off) for 5 min. The solutions were centrifuged at 11,000×g for 30 min and filtered through a 0.2 μm filter. Each sample (10 μL) was added to 1 mL ethanol and measured by absorption via UV-Vis spectroscopy. The extinction coefficients were 9970 M⁻¹cm⁻¹, 26343 M⁻¹cm⁻¹, and 13476 M⁻¹cm⁻¹ for cycloretinal, 2,4-dimethylbenzaldehyde, and β-ionone, respectively. Due to a lack of absorption characteristic, approximate concentrations for glyoxal, methylglyoxal, and β-cyclocitral were used (amounts shown above).

### Example 8

### In Vivo Analysis of Lipofuscin Cytotoxicity

### Retinal Cell Growth

Retinal Pigment Epithelial Cells (ARPE-19 from CRL-2302, American Type Culture Collection (ATCC)) were grown in Dulbecco's Modified Eagle medium/nutrient mixture F12 (DMEM/F12) with 10% fetal bovine serum in accordance with the manufacturer's protocols. Cells were cultured at 37° C. under 5% CO₂ with 1% gentamycin (50 mg/mL). Cells were cultured to near confluency and harvested with Trypsin/EDTA after being washed with Dulbecco's Phosphate Buffered Saline.

### Evaluating Cytotoxicity of Cycloretinal, β-Ionone, 2,4-Dimethylbenzaldehyde, Glyoxal, Methylglyoxal, and β-Cyclocitral

ARPE-19 cells were grown to confluency in 24-well plates with 900 μL DMEM/F12 (10% FBS). Aliquots (100 μL) of the cycloretinal liposomal complexes were applied in triplicate to the 24-well plates at various concentrations (0-206 μM) and the cells were incubated for 24 hours. Using only media and water, controls were generated for each plate. The same conditions were used to assess the cytotoxicity of β-Ionone, 2,4-dimethylbenzaldehyde, glyoxal, methylglyoxal, and β-cyclocitral, respectively. The range of concentrations used for β-Ionone was 0-167 μM and the range for 2,4-dimethylbenzaldedhyde was 0-151 μM. The range of concentrations for glyoxal, methylglyoxal, and β-cyclocitral were 0-1000 μM, respectively. Vehicle controls were prepared by delivering maximum volume of liposomes that was respectively delivered for each substrate prepared (e.g. highest concentration of cycloretinal required 44 μL of liposomal encapsulated cycloretinal. Therefore, 44 μL of liposomal vehicle control was delivered separately to retinal cells in triplicate).

Following 24 hour incubation with the liposomal complexes, light induced cell damage was measured by exposing cells to 10 min of irradiation (70 W lamp) through a 430 nm bandpass filter (Thor labs, 430±2 nm). The cells were cultured for an additional 72 hours, the media was removed, and the plates were washed three times with DPBS and resuspended in 200 μL of buffer.

To evaluate non-light induced toxicity, near confluent cells, in 900 μL media, were exposed to the same concentrations of liposomal complexes and allowed to incubate for 96 hours. The cells were then washed three times with DPBS and resuspended in 200 μL of buffer.

### MTT Assay

Cell viability was assessed with the MTT cell proliferation assay. The MTT assay utilizes a yellow tetrazolium, MTT (3-(4, 5-dimethylthiazolyl-2)-2,5-diphenyltetrazolium bromide), which is reduced by metabolically active cells. The viability was measured following the manufacturer's suggested protocol. The MTT reagent was added in 10 μL aliquots and incubated at 37° C. under 5% CO₂ for 4 hours. The detergent reagent (100 μL) was added to each well and was cultured in the dark at room temperature for 2 hours. The absorbance was subsequently evaluated at 570 nm utilizing a microplate reader (quant absorbance plate reader, BioTek Instruments Inc.)

### Example 9

### Degradation Product Analysis

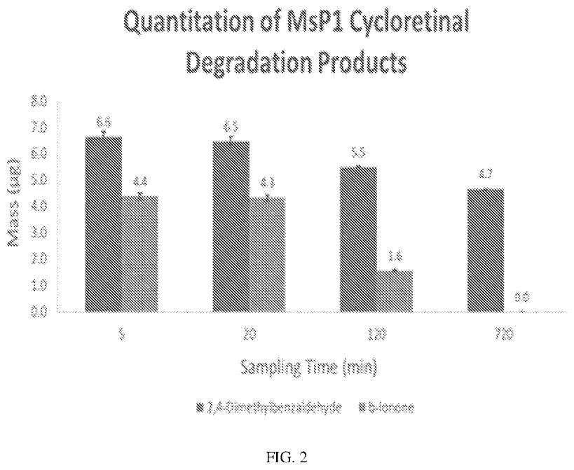

To identify the degradation products, cycloretinal was incubated for 1 hour with MsP1 in the presence of hydrogen peroxide. Following incubation, the reaction mixture was extracted and analyzed by GC-MS. While cycloretinal was not isolated, 4 peaks were observed with retention times (RT) of 12.12, 14.01, 14.07 and 19.07 min, respectively (FIG. 2). The peaks at 12.12 and 14.01 min were characterized as 2,4-dimethylbenzaldehyde (2,4-DMB) and β-ionone respectively by comparison of fragmentation data from GC-MS analysis with those of synthetic standards. The peaks at 14.07 and 19.07 min were not detectable by HPLC, suggesting that these compounds either lacked a chromophore or were too volatile to be detected. Time course analysis of reaction samples were performed by HPLC (FIG. 2).

Quantification of peaks by standard curve analysis showed that (3-ionone decreases to undetectable levels by 12 hours. However, the rate of 2,4-DMB degradation is slower and still remains at 12 hours. Due to the volatile nature of some species observed by GC-MS, aminoguanidine (AG) was used to probe whether any α-oxoaldehydes (glyoxal and methylglyoxal) formed from the enzymatic degradation of cycloretinal. After overnight incubation of cycloretinal with MsP1 in the presence of hydrogen peroxide, enzyme was removed by 10 kDa PES filtration. The aqueous phase was incubated with aminoguanidine (37° C. for 2 h) prior to extraction of products in chloroform. The combined organic phases were concentrated in vacuo and resuspended in methanol and submitted for LC-MS and LC-MSMS analysis.

LC-MS analysis was able to confirm the formation of 3-amino-1,2,4-triazine derivatives that formed from the coupling reaction of α-oxoaldehydes (glyoxal and methylglyoxal). LC-MS/MS fragmentation patterns observed are shown in Table 2.

TABLE 2

| | | | MsP1 Cyclo AG (Observed) | | |
|---|---|---|---|---|---|
| | Theoretical | | | | Intensity |
| | m/z | Intensity | m/z | Intensity | Corrected |
| Scan m/z 97 | 97.051 | 100 | 97.051 | 20734.52 | 100 |
| (Parental Ion) | 70.040 | 18.80 | 70.040 | 3069.88 | 14.81 |
| Scan m/z 111 | 111.067 | 100 | 111.067 | 235984.63 | 100.00 |
| (Parental Ion) | 84.056 | 0.46 | 84.056 | 2838.63 | 1.20 |
| | 70.040 | 16.71 | 70.040 | 1592.35 | 0.67 |
| Scan m/z 153 | 153.127 | 100 | 153.127 | 15549.43 | 100 |
| (Parental Ion) | 135.117 | 12.12 | 135.117 | 1589.79 | 10 |
| | 125.096 | 1.36 | 125.096 | 2022.11 | 13 |
| | 109.101 | 1.28 | 109.101 | 4596.82 | 30 |
| Scan m/z 205 | 205.086 | 100 | 205.086 | 8141.85 | 100 |
| (Parental Ion) | 177.091 | 8 | 177.091 | 5000.57 | 61 |
| | 163.075 | 9 | 163.075 | 3407.14 | 42 |
| | 159.08 | 2 | 159.081 | 7932.66 | 97 |
| Scan m/z 231 | 231.101 | 100 | 231.101 | 8465.4 | 100 |
| (Parental Ion) | 213.091 | 72 | 213.091 | 1260.56 | 15 |
| | 187.075 | 37 | 187.075 | 1132.71 | 13 |

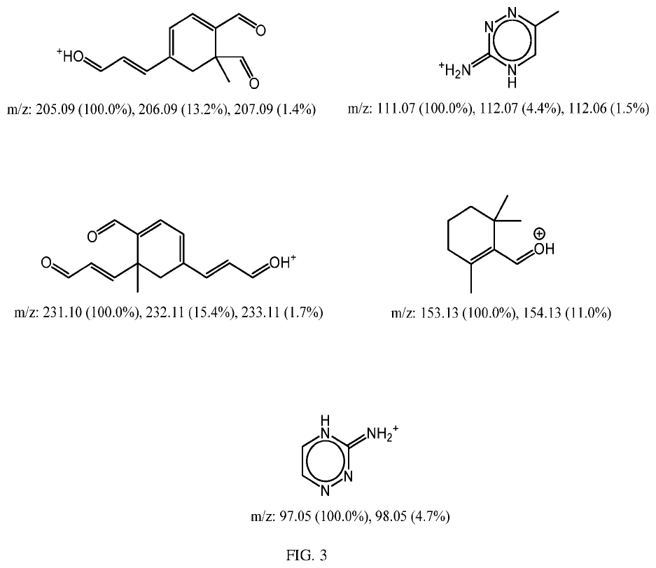

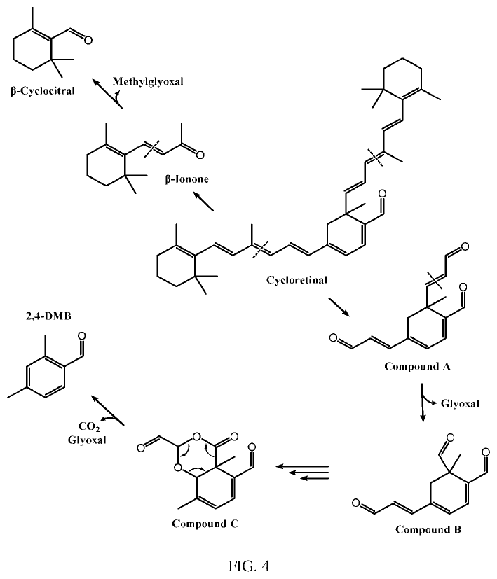

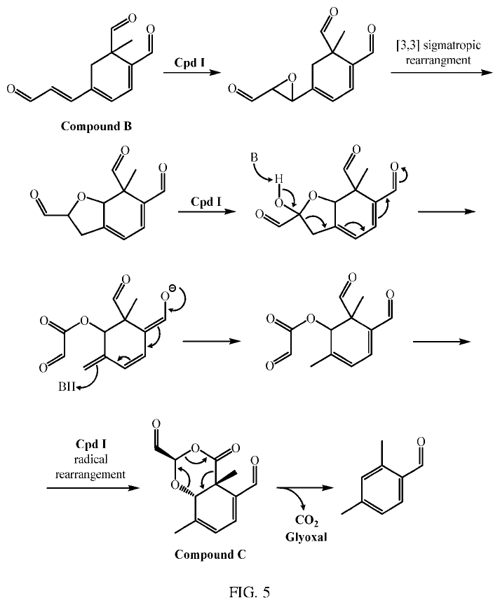

Cycloretinal degradation in the presence of MsP1 produced 5 distinct fragments (FIG. 3). These fragments support the production of glyoxal, methylglyoxal, β-cyclocitral, and 2,4-dimethylbenzaldehyde as shown in FIG. 4. MsP1-catalyzed oxidative degradation of cycloretinal to produce compound B (FIG. 4) follows a similar mechanism as the HRP-catalyzed degradation of A2E. The activity of MsP1 reacting with compound B utilizes epoxidation, hydroxylation, as well as a combination of rearrangements to yield a thermodynamically favorable product, 2,4-DMB, with the release of carbon dioxide (FIG. 5).

### Example 10

### Toxicity Analysis

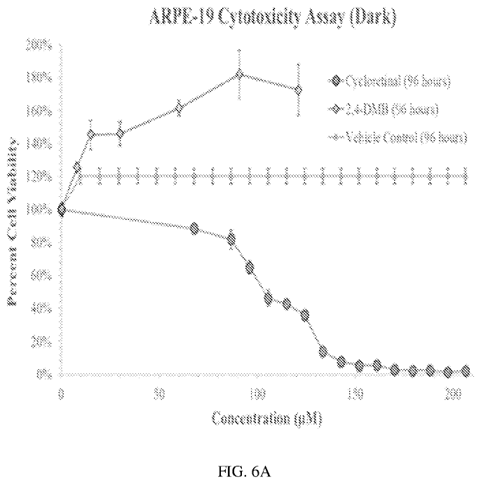

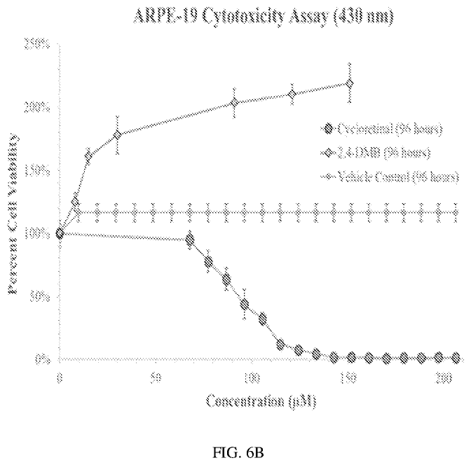

The instant example evaluates the cytotoxicity of cycloretinal and its degradation products following MsP1 degradation. Liposomal capsules of 2,4-DMB, β-ionone, glyoxal, methylglyoxal, and β-cyclocitral were generated and incubated with ARPE-19 cells for 24 hours. The cells were exposed to 430 nm radiation and subsequently incubated in the dark (FIG. 6A). The experiment was repeated without exposure to 430 nm radiation to evaluate non-light-induced cytotoxicity (FIG. 6B). Cell viability was measured as a function of NADPH dependent oxidoreductase activity using the MTT (3-[4,5-dimethylthiazol-2-yl]-2,5 diphenyl tetrazolium bromide) assay.

Cycloretinal displayed slight enhancement in cytotoxicity under light conditions (EC₅₀=93±6.1 μM) versus dark (EC₅₀=104±2.2 μM). Although observed cytotoxicity of cycloretinal is relatively high, studies have shown that only a fraction of the pigment delivered (˜30% over 14 days) resides within cells. Similarly, an exponential decay correlation between pigment concentration and days until cell death was observed, where lower concentration of pigment required longer incubation time before cell death was observed.

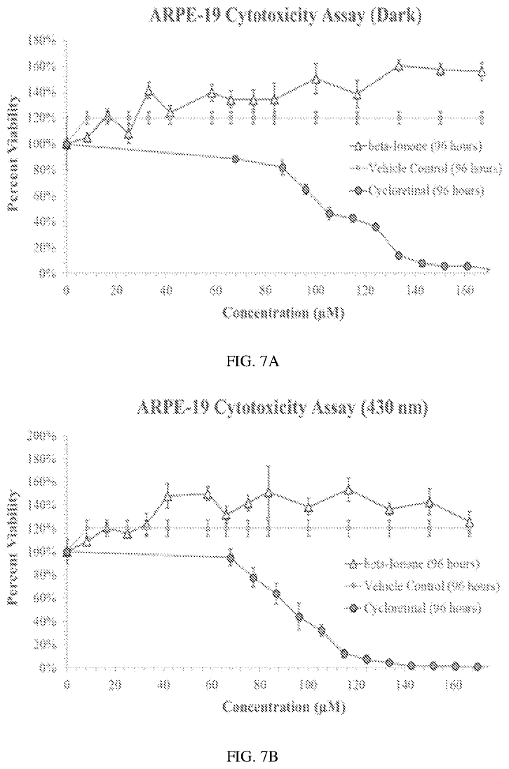

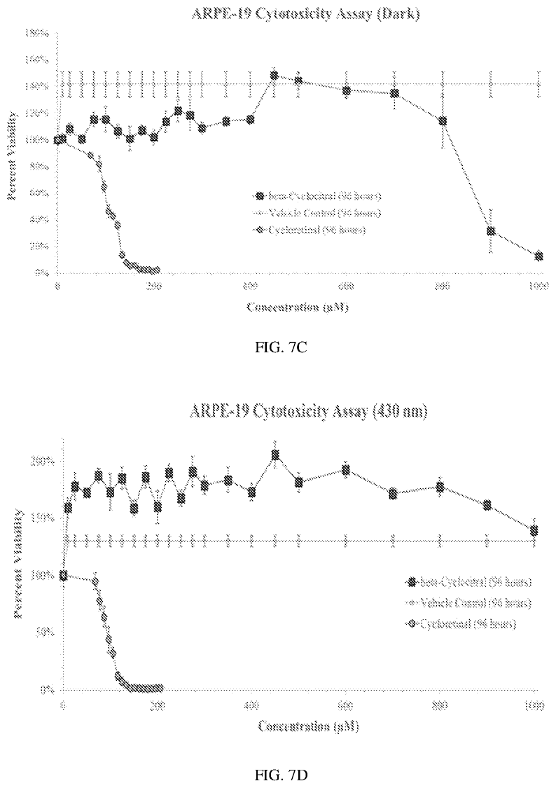

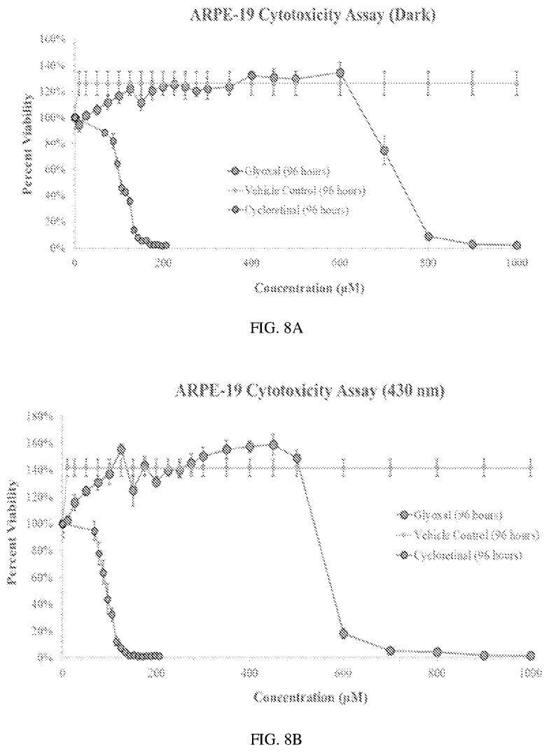

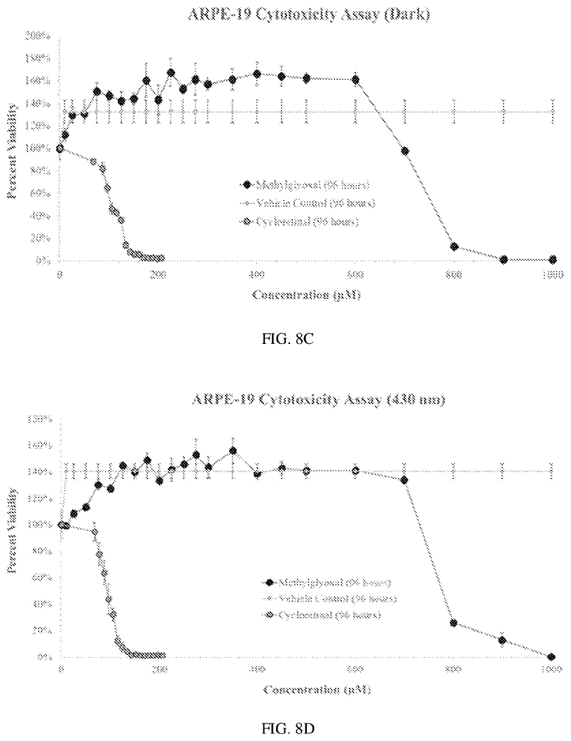

In contrast, 2,4-DMB, β-ionone, glyoxal, methylglyoxal, and β-cyclocitral seemed to have no impact on cell viability at similar concentrations over a 96-h period when compared with cycloretinal (see FIGS. 7A-7D and 8A-8D). 2,4-DMB promoted cell growth, which might suggest its use as a carbon source. Likewise, results do not suggest that light exposure had a marginal effect on cell viability of ARPE-19 cells exposed to cycloretinal or β-ionone, there seems to be a slight enhancement in cell viability of ARPE-19 cells exposed to 2,4-DMB with light exposure. This might suggest that 2,4-DMB oxidizes in the presence of light, facilitating the breakdown and uptake of these oxidized byproducts.

### Example 11

### Kinetic and Activity Analysis

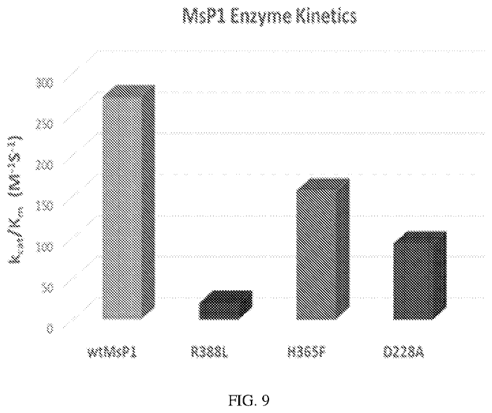

Kinetics of MsP1 catalyzed degradation of cycloretinal was obtained by monitoring the decrease in its absorbance at 430 nm. The molar absorptivity co-efficient for cycloretinal was calculated and utilized to assess concentration. The k_{cat}/Kₘ for MsP1 catalyzed degradation of cycloretinal was calculated as 271 M⁻¹s⁻¹ by Lineweaver Burke analysis. The k_{cat}/Kₘ was found to be comparable to the kinetics of anthraquinone degradation by Class B enzymes of the DyP superfamily but several orders less than the k_{cat}/Kₘ for other Class D enzymes. Without being bound by any theory, this can indicate that the measure of only peroxidase activity in the case of other Class D enzymes, whereas here complete degradation of cycloretinal is measured. A study of the effect of pH on catalytic activity indicated that a pH of 5.5 was optimal for MsP1 catalytic activity towards cycloretinal (FIG. 9).

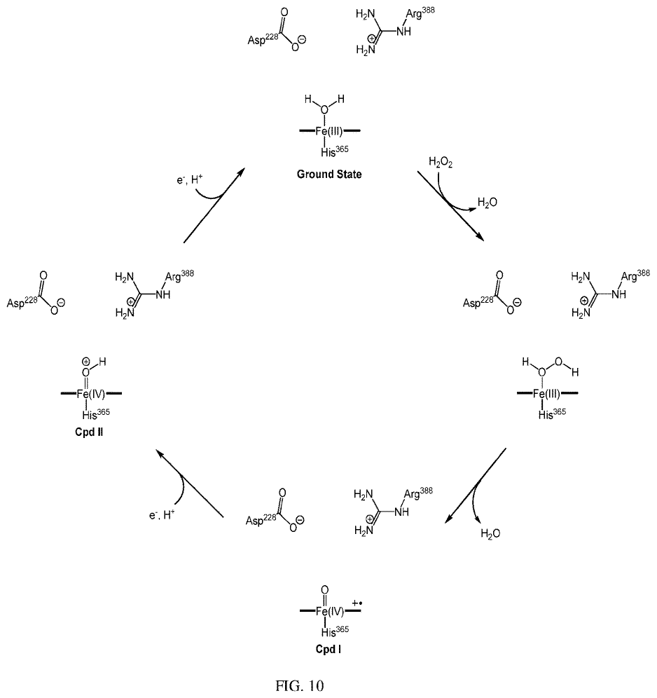

Active site residues were selected based on multiple sequence alignment with homologous DyPs and protein modelling, His 365, Asp 228, and Arg 388 were identified as amino acids that are critical to MsP1 activity. D228A, H365F, and R388L variants were generated, and their activity was determined to be significantly reduced compared to that of the wild-type protein. The R388L variant was found to be about fourteen times less active than the wild-type MsP1, whereas H365F and D228A were about 1.7 to 2.9-fold less active than wild-type MsP1 (FIG. 9). Interestingly, D228A variant of MsP1 had the greatest enzyme turnover number (k_{cat}), however the enzyme-substrate affinity is less than wtMsP1 since the Kₘ value for D228A is greater than wtMsP1. This means that wtMsP1 has greater substrate affinity for cycloretinal, compared with D228A and R388L variants, and reaches half maximal velocity at lower substrate concentration. The Michaelis-Menten constant (K₁) for H365F variant of MsP1 is the lowest of all proteins assayed by steady-state kinetics, which demonstrates slightly greater enzyme-substrate affinity compared with wtMsP1. The greater affinity observed for the phenylalanine variant likely signifies that cycloretinal can dock with greater affinity in the active site since the iron porphyrin heme cofactor would no longer be covalently bound at residue 365. Therefore, the residues involved in MsP1 catalysis are D228, H365, and R388 (FIG. 10).

Table 3 shows the kinetic constants of wild-type MsP1 and single variants oxidizing cycloretinal (means and 95% confidence limits):

TABLE 3

| | | |
|---|---|---|
| wtMs P1 | k_{cat}/Kₘ (M⁻¹S⁻¹) | 271 ± 6.2 |
| | k_{cat} (Sec-1) | 2.29E−01 ± 2.4E−03 |
| | Kₘ (M) | 8.45E−04 ± 1.1E−05 |
| R388L | k_{cat}/Kₘ (M⁻¹S⁻¹) | 19.2 ± 0.3 |
| | k_{cat} (Sec-1) | 2.18E−02 ± 1.8E−04 |
| | Km (M) | 1.14E−03 ± 1.1E−05 |
| H365F | k_{cat}/Kₘ (M⁻¹S⁻¹) | 156 ± 8.0 |
| | kcat (Sec-1) | 1.42E−01 ± 3.1E−03 |
| | Km (M) | 9.06E−04 ± 2.6E−05 |
| D228A | k_{cat}/Kₘ (M⁻¹S⁻¹) | 91.6 ± 3.5 |
| | k_{cat} (Sec-1) | 3.88E−01 ± 7.3E−03 |
| | Kₘ (M) | 4.23E−03 ± 8.2E−05 |

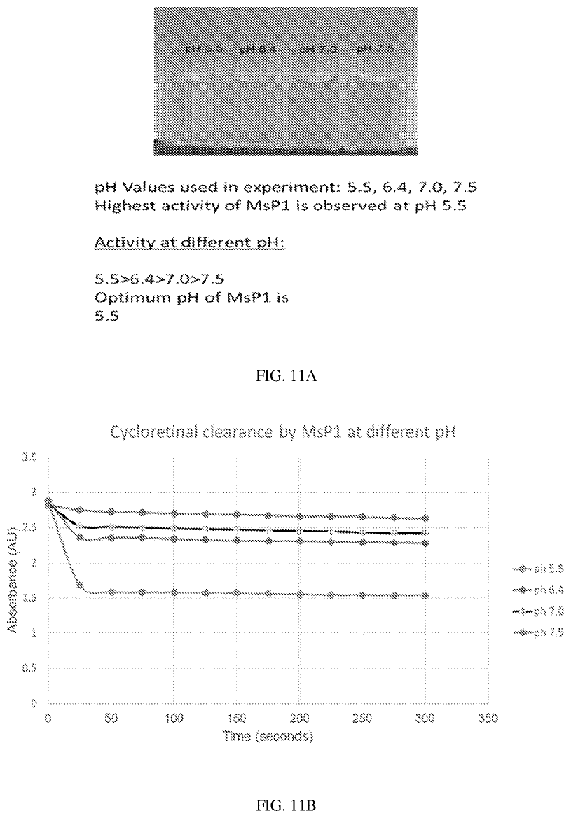

Enzymes that degrade cycloretinal show promise as a gene therapeutic to alleviate the build-up of lipofuscins, thereby reducing the risk of AMD progression.¹¹ An isolated enzyme, MsP1, was studied for its natural affinity for cycloretinal (k_{cat}/Kₘ of 271 M⁻¹s⁻¹) and is highly active at lysosomal pH of 5.5 (FIGS. 11A-11B). Bis-retinoids are believed to be mainly present in the lysosomes of RPE.¹¹ The degradation compounds, 2,4-DMB, β-ionone, glyoxal, methylglyoxal, and β-cyclocitral do not exhibit toxicity to ARPE-19 cells unlike cycloretinal (dark EC₅₀=104±2.2 μM). With a future goal in mind to engineer a more efficient enzyme with increased specificity towards cycloretinal, mutagenesis studies were employed to understand the residues involved in peroxidase activity. An arginine believed to be involved in formation of Cpd I was shown to be more critical for catalytic activity in comparison to the heme chelating histidine (H365). Rational design and/or random mutagenesis approaches might aid in improving the catalytic competency of the enzyme for future development of MsP1 as a gene-based strategy to catabolize harmful lipofuscins and impede the progression of dry AMD.

1. A pharmaceutical composition comprising i) a MsP1 enzyme and ii) one or more pharmaceutically acceptable carriers.

2. The pharmaceutical composition of claim 1, wherein the MsP1 enzyme comprises an amino acid sequence encoding an MsP1 protein.

3. (canceled)

4. The pharmaceutical composition of claim 2, wherein the amino acid sequence comprises at least 90% sequence identity to the sequence of SEQ ID NO: 1.

5. (canceled)

6. The pharmaceutical composition of claim 2, wherein the amino acid sequence comprises an aspartate (D) amino acid at position 228 of SEQ ID NO: 1, or wherein the amino acid sequence comprises a histidine (H) amino acid at position 365 of SEQ ID NO: 1, or wherein the amino acid sequence comprises an arginine (R) amino acid at position 388 of SEQ ID NO: 1.

7. (canceled)

8. (canceled)

9. The pharmaceutical composition of claim 1, wherein the MsP1 enzyme comprises a nucleic acid sequence encoding an MsP1 protein.

10. (canceled)

11. The pharmaceutical composition of claim 9, wherein the nucleic acid sequence comprises at least 90% sequence identity to the sequence of SEQ ID NO: 2.

12. (canceled)

13. The pharmaceutical composition of claim 9, wherein the nucleic acid sequence provides for an aspartate (D) amino acid at position 228 of the amino acid sequence of SEQ ID NO: 1, or wherein the amino acid sequence comprises a histidine (H) amino acid at position 365 of SEQ ID NO: 1, or wherein the amino acid sequence comprises an arginine (R) amino acid at position 388 of SEQ ID NO: 1.

14. (canceled)

15. (canceled)

16. (canceled)

17. The pharmaceutical composition of claim 9, wherein the nucleic acid sequence comprises at least 90% sequence identity to the sequence of SEQ ID NO: 3.

18. (canceled)

19. The pharmaceutical composition of claim 9, wherein the nucleic acid sequence provides for an aspartate (D) amino acid at position 228 of the amino acid sequence of SEQ ID NO: 1, or wherein the amino acid sequence comprises a histidine (H) amino acid at position 365 of SEQ ID NO: 1, or wherein the amino acid sequence comprises an arginine (R) amino acid at position 388 of SEQ ID NO: 1.

20. (canceled)

21. (canceled)

22. The pharmaceutical composition of claim 9, wherein the nucleic acid sequence is a native sequence that has been modified to comprise codons optimized for expression in prokaryotic cells.

23. (canceled)

24. The pharmaceutical composition of claim 22, wherein the nucleic acid sequence comprises at least 90% sequence identity to the sequence of SEQ ID NO: 4.

25. (canceled)

26. The pharmaceutical composition of claim 22, wherein the nucleic acid sequence provides for an aspartate (D) amino acid at position 228 of the amino acid sequence of SEQ ID NO: 1, or wherein the amino acid sequence comprises a histidine (H) amino acid at position 365 of SEQ ID NO: 1, or wherein the amino acid sequence comprises an arginine (R) amino acid at position 388 of SEQ ID NO: 1.

27. (canceled)

28. (canceled)

29. A method of treating an eye disorder in a patient in need thereof, said method comprising the step of administering a therapeutically effective amount of a pharmaceutical composition to the patient, wherein the pharmaceutical composition comprises i) a MsP1 enzyme and ii) one or more pharmaceutically acceptable carriers.

30. The method of claim 29, wherein the eye disorder is age-related macular degeneration (AMD).

31. The method of claim 30, wherein the AMD is dry form AMD.

32. The method of claim 30, wherein the AMD is wet form AMD.

33. The method of claim 29, wherein the administration provides a reduction in one or more symptoms of the eye disorder in the patient.

34. (canceled)

35. (canceled)

36. (canceled)

37. (canceled)

38. The method of claim 29, wherein the administration degrades cycloretinal in the patient.

39. The method of claim 29, wherein the administration provides a reduction in clinical progression of the eye disorder in the patient.

40. (canceled)

41. A method of reducing cycloretinal in a patient in need thereof, said method comprising the step of administering a therapeutically effective amount of a pharmaceutical composition to the patient, wherein the pharmaceutical composition comprises i) a MsP1 enzyme or a variant thereof and ii) one or more pharmaceutically acceptable carriers.

42. (canceled)

43. (canceled)

44. (canceled)

45. (canceled)


SEQUENCE LISTING (10 sequences)

SEQ ID NO: 1 - protein, 513 aa, Mycetinis scorodonius
  REGION 1..513: note = MsP1 amino acid sequence
MKLFSASVFAAIIASHYASATAHIRAPNVKPRRTNSLLTAPPQQPPLPSAQQAASASSSAGLNLTDIQGDILIGMKKNKELFFFFSITDAATFKAKLGSDILELITSTNQLLAVATQPITAVNVAFSSTGLKALGITDDLKDPVFEAGMLSNAVSDLSDPGTGNWVPGFVGTSVHGVFLLASDTIDNVNTELANIQTILNGSITEIHRLQGEARPGDQQGHEHFGFMDGISNPAVDGFTPPAEIRPGQALIPPGIMLLGEANDTFQNDRPPWAKDGSFLVFRQMQQRAPEFNKFLQDHALNMPNMTSEQGADLLGARIVGRWKSDAPIDLTPLVDDPVLAADNQRNNNFDFSDATNQTRCPFSAHIRKANPRGDLGGINKFPNQHIIRAGIPYGPEVTDAEKASNSSSTDPSLERGLAFVAYQSNIQNGFVFLQKNWVDNTNFFRPGTGVDPLIGTNSRNSGTDAPNTPRVVSGLDPNNATSTIEIGIDFVVSRGGEYFFSPSLSAIRTVLSV

SEQ ID NO: 2 - DNA, 2093 bp, Mycetinis scorodonius
  source 1..2093: mol_type = genomic DNA
  misc_feature 1..2093: note = full MsP1 nucleotide sequence
atgaagcttttttctgcctccgtttttgctgctatcatcgctagtcactatgcgtcagcgactgcccacatcagggctcccaacgtgaagccaaggaggacaaactcacttctgactagtgagagcgttccactcctttccaaagtagttgcttatcggggtttcgaaattctctagctccccctcaacagcctccgcttccatctgctcaacaggctgcaagtgcctctagcagtgctggcttgaatctcaccgacatccagggtgatattctgtacgtatctttcgatatttcttgtgtttatcgcagctaatgtacctgtttcaggatcggcatgaagaagaacaaggaactatttttcttcttcagcatcaccgacgccgctactttcaaggtatttgcttttatatctgtatttcgaacatattttcacagttaatttcaaggctaagctaggatccgacattcttgaactaatcacatcgaccaatcagttactcgccgtcgccactcagcctatcacggctgtcaacgtcgctttctctagcactggcctcaaggcattgggtatcacagatgatctgaaggatcctgtcttcgaggccggaatgctcagcaacgcagtgagcgacttgagcgatccagggaccggcaattgggtgcctgggtttgtcggcaccagtgttcatggcgttttcctacttgcatcggacaccattgacaatgtaaacaccgagctggccaacatccaaaccattttgaatggctcgatcacggagattcatcgtttgcaaggggaggctcgacccggtgaccagcaaggtcacgaacgtaagatatttgtttcccgctccgtactctgctcatctgcatatcgacagactttggattcatggatggaatcagtaacccggccgttgatggatttacacctccagcggaaataagacctggacaagctttaattccgcctggtatcatgcttctcggagaggcaaacgacacttttcagaatgatcgtcctccgtgggccaaagatggttccttccttgtcttccgtcaaatgcaacagcgcgcgcccgagttcaacaagttcctgcaagatcacgctcttaacatgccgaatatgacatccgagcaaggcgctgatctccttggtgccaggattgtaggacgatggaaaagtgtaagctctctcttcgtatagatggcatcctactcagctcaacgttcggtggttaggatgctcctattgacctcactccgttggtcgatgacccagtgttggctgctgacaatcagcgaaataacaacttcgacttttctgacgccacgaatcagacacgttgccctttctctgctcatatccgcaaggctaacccgcgcggtgatcttgggggtattaataaattcccaaaccaacacataatccgagcgggaattccgtatggacccgaaggtgccatttattctgcatcgtccctgcagcataattctgattccttctttttggttcagttaccgacgctgaaaaagcgtcaaatagctctagcactgaccctagtctggagcgtggtctggcgtttggtgagtagaaagccctatttagagcgacaggcctaatttggaagattctagtggcctatcagtctaatatccagaacggattcgtattccttcaaaagaattgggttgataatacgaagtgcgttctgccgaattctgtcctgcagcttgaaactaatcacattgtaaaaacgcagtttcttccgacccggcactggtgtagatcctctcatcggtaatgcagctgcattcgaagaaagccctcctagtcgttgctaatgaatacgtttttaggtacaaattctcgtaacagtggcaccgatgcccccaacacgcctcgtgtcgtcagcggcttggatcctaataacgctacgagcaccatcgaaattggtatcggtatgtgtatccgcttcctatgaaacttacattcccgctaaccctgtgctcagatttcgtagtttctcgtggaggagaatacttcttctcgccctcactttctgcgatcaggactgtgctttcagtctag

SEQ ID NO: 3 - DNA, 1539 bp, Mycetinis scorodonius
  source 1..1539: mol_type = genomic DNA
  misc_feature 1..1539: note = MsP1 nucleotide sequence
atgaagcttttttctgcctccgtttttgctgctatcatcgctagtcactatgcgtcagcgactgcccacatcagggctcccaacgtgaagccaaggaggacaaactcacttctgactgctccccctcaacagcctccgcttccatctgctcaacaggctgcaagtgcctctagcagtgctggcttgaatctcaccgacatccagggtgatattctgatcggcatgaagaagaacaaggaactatttttcttcttcagcatcaccgacgccgctactttcaaggctaagctaggatccgacattcttgaactaatcacatcgaccaatcagttactcgccgtcgccactcagcctatcacggctgtcaacgtcgctttctctagcactggcctcaaggcattgggtatcacagatgatctgaaggatcctgtcttcgaggccggaatgctcagcaacgcagtgagcgacttgagcgatccagggaccggcaattgggtgcctgggtttgtcggcaccagtgttcatggcgttttcctacttgcatcggacaccattgacaatgtaaacaccgagctggccaacatccaaaccattttgaatggctcgatcacggagattcatcgtttgcaaggggaggctcgacccggtgaccagcaaggtcacgaacactttggattcatggatggaatcagtaacccggccgttgatggatttacacctccagcggaaataagacctggacaagctttaattccgcctggtatcatgcttctcggagaggcaaacgacacttttcagaatgatcgtcctccgtgggccaaagatggttccttccttgtcttccgtcaaatgcaacagcgcgcgcccgagttcaacaagttcctgcaagatcacgctcttaacatgccgaatatgacatccgagcaaggcgctgatctccttggtgccaggattgtaggacgatggaaaagtgatgctcctattgacctcactccgttggtcgatgacccagtgttggctgctgacaatcagcgaaataacaacttcgacttttctgacgccacgaatcagacacgttgccctttctctgctcatatccgcaaggctaacccgcgcggtgatcttgggggtattaataaattcccaaaccaacacataatccgagcgggaattccgtatggacccgaagttaccgacgctgaaaaagcgtcaaatagctctagcactgaccctagtctggagcgtggtctggcgtttgtggcctatcagtctaatatccagaacggattcgtattccttcaaaagaattgggttgataatacgaatttcttccgacccggcactggtgtagatcctctcatcggtacaaattctcgtaacagtggcaccgatgcccccaacacgcctcgtgtcgtcagcggcttggatcctaataacgctacgagcaccatcgaaattggtatcgatttcgtagtttctcgtggaggagaatacttcttctcgccctcactttctgcgatcaggactgtgctttcagtc

SEQ ID NO: 4 - DNA, 1539 bp, synthetic construct
  source 1..1539: mol_type = other DNA
  misc_feature 1..1539: note = codon optimized nucleotide sequence
atgaaactgttctccgcatcggtctttgcagccatcatcgcttctcactacgcctcagccacggcacacatccgcgctccgaatgttaaaccgcgtcgcaccaacagcctgctgacggcaccgccgcagcaaccgccgctgccgagcgcacagcaagcggcctctgcgagctctagtgccggtctgaatctgaccgatatccagggcgacattctgatcggtatgaagaaaaacaaagaactgtttttctttttctcaattaccgatgcagctacgtttaaagcgaaactgggcagtgacatcctggaactgattaccagcaccaatcagctgctggcagtggctacccaaccgatcacggcagttaacgtcgctttttcctcaaccggcctgaaagccctgggtattacggatgacctgaaagatccggtcttcgaagcgggtatgctgagcaatgccgtgagtgatctgtccgacccgggcaccggtaactgggtcccgggctttgtgggtacgagcgtgcatggcgttttcctgctggcgtctgataccattgacaatgtgaacacggaactggccaatatccaaaccattctgaacggtagtatcacggaaattcaccgtctgcagggtgaagcacgtccgggtgatcagcaaggtcatgaacactttggcttcatggatggtatctccaacccggcagttgacggttttaccccgccggcagaaattcgtccgggtcaggctctgatcccgccgggtattatgctgctgggtgaagcaaatgataccttccagaacgaccgcccgccgtgggctaaagatggtagctttctggtgttccgtcagatgcagcaacgcgcaccggaatttaataaattcctgcaagatcatgctctgaatatgccgaacatgaccagcgaacagggtgcggatctgctgggtgcacgtatcgttggccgctggaaatctgatgcgccgattgacctgaccccgctggttgatgacccggtcctggcagccgataaccaacgtaacaacaacttcgatttctcagacgccaccaaccagacgcgctgcccgttttcggcacatatccgtaaagctaatccgcgcggcgatctgggcggtattaataaattcccgaaccagcacattatccgtgcgggcattccgtatggtccggaagttaccgatgcggaaaaagcctcaaattcgagctctacggacccgtcgctggaacgcggtctggcatttgtcgcttaccagagtaatatccaaaacggctttgtgttcctgcagaaaaactgggttgataataccaactttttccgtccgggcacgggtgttgacccgctgattggcaccaacagccgtaacagcggcaccgatgcaccgaatacgccgcgcgtggttagcggtctggacccgaataacgcgaccagcaccattgaaatcggcattgattttgtcgtgtctcgtggcggcgaatacttcttctccccgtccctgtcggctatccgtaccgtcctgtccgtt

SEQ ID NO: 5 - DNA, 40 bp, synthetic construct
  source 1..40: mol_type = other DNA
  misc_feature 1..40: note = BamHI-MsP1-F
ggatcccaccaccaccaccaccacaaactgttctccgcat

SEQ ID NO: 6 - DNA, 34 bp, synthetic construct
  source 1..34: mol_type = other DNA
  misc_feature 1..34: note = HindIII-MsP1-R
aagcttttaaacggacaggacggtacggatagcc

SEQ ID NO: 7 - DNA, 41 bp, synthetic construct
  source 1..41: mol_type = other DNA
  misc_feature 1..41: note = H221A MsP1-F
tccgggtgatcagcaaggtgcagaacactttggcttcatgg

SEQ ID NO: 8 - DNA, 41 bp, synthetic construct
  source 1..41: mol_type = other DNA
  misc_feature 1..41: note = H221A MsP1-R
ccatgaagccaaagtgttctgcaccttgctgatcacccgga

SEQ ID NO: 9 - DNA, 30 bp, synthetic construct
  source 1..30: mol_type = other DNA
  misc_feature 1..30: note = D336A MsP1-F
cccgctggttgatgcaccggtcctggcagc

SEQ ID NO: 10 - DNA, 30 bp, synthetic construct
  source 1..30: mol_type = other DNA
  misc_feature 1..30: note = D336A MsP1-R
gctgccaggaccggtgcatcaaccagcggg